(19) United States
(12) Patent Application Publication
(10) Pub. No.: US 2024/0091109 A1
(43) Pub. Date: Mar. 21, 2024

(54) COMPOSITION COMPRISING POLYMERS OF NATURAL ORIGIN AND HAVING PROPERTIES FOR THICKENING POLAR MEDIA

(71) Applicant: Societe d'Exploitation de Produits Pour les Industries Chimiques Seppic, PARIS CEDEX 07 (FR)

(72) Inventors: Miruna BODOC, CASTRES (FR); Stéphane MONTEILLET, CASTRES (FR)

(21) Appl. No.: 18/260,177

(22) PCT Filed: Dec. 22, 2021
(86) PCT No.: PCT/EP2021/087339

§ 371 Date: Jun. 30, 2023

(30) Foreign Application Priority Data
Jan. 7, 2021 (FR) 2100108

(51) Int. Cl. A61K 8/06; A61K 8/37; A61K 8/44; A61K 8/49; A61K 8/88; A61Q 19/00; C08G 69/10; C08K 5/103; C08K 5/1515

(52) U.S. Cl. CPC A61K 8/064; A61K 8/375; A61K 8/44; A61K 8/4973; A61K 8/88; A61Q 19/00; C08G 69/10; C08K 5/103; C08K 5/1515; A61K 2800/48; A61K 2800/805; A61K 2800/95

(57) ABSTRACT

Composition (CA) in the form of a self-reversible water-in-oil-type emulsion comprising, for 100% of its mass, a mass content of greater than or equal to 20% of a polymer (P) consisting of monomeric units derived from glutamic acid (GA), partially or totally salified, and units derived from at least one crosslinking agent (AR) bearing at least two glycidyl functions.

The present invention relates to a composition in the form of a self-inverting water-in-oil emulsion, to the process for preparing such a composition, and to the use of this composition for preparing cosmetic compositions for topical use.

Polymers are today widely used in cosmetic formulations for topical use and represent the second-most widely used family of products in complex formulations of this type. Cosmetic compositions contain polar phases, for example phases consisting of water, and in most cases require the use of rheology modifiers, for example polymers, to increase the viscosity of these polar phases, and also to impart well-defined rheological behavior.

Polymers that modify the rheology of polar phases include natural polymers, for example polysaccharides based on saccharides or polysaccharides based on saccharide derivatives, or else synthetic polymers of linear or branched, crosslinked or noncrosslinked, anionic or cationic or amphiphilic polyelectrolyte type. Predominantly marketed as ingredients intended for cosmetic formulations, synthetic polymers have the property of spreading out in the polar phase as a result of electrostatic repulsions caused by the presence of charges (negative and/or positive) on the linear or branched, crosslinked or non-crosslinked polymer backbone. These rheology-modifiers provide both an increase in the viscosity of the polar phase and also a degree of consistency and/or a stabilizing effect conferred on the cosmetic, dermocosmetic or dermopharmaceutical formulation to be thickened.

To meet consumer needs and to improve the performance of cosmetic formulations, various recent scientific studies have reported the development of novel, innovative and varied polymer systems. Thus, polymers used in the cosmetics or dermocosmetics industries can play a functional role as film-forming agents, rheology modifiers, agents permitting the stabilization of fatty phases in water-in-oil and oil-in-water emulsions or the stabilization of solid particles (pigments or fillers), or even as agents conferring particular sensory properties (softness to the touch, ease of handling and application, freshness effect, etc.) or as agents also having a direct impact on the appearance of the formula (transparent, translucent or opaque).

Polymers that modify the rheology of polar phases, and more particularly of aqueous phases, are mainly polyelectrolytes resulting from the free-radical polymerization of (meth)acrylic monomers, i.e. acrylic acid or methacrylic acid, ester derivatives of acrylic acid or methacrylic acid, or else derivatives of acrylamide or methacrylamide.

Developing novel biobased and biodegradable rheology modifiers that are as effective as the synthetic polymers in current use still constitutes a major challenge and a key issue for suppliers of cosmetic ingredients. Specifically, the solutions mostly employed for thickening aqueous phases have up to now involved ingredients originating from raw materials of petrochemical origin, notably acrylic acid and derivatives thereof or methacrylic acid and derivatives thereof.

Given the growing consumer concern for a sustainable and responsible economy and development, the replacement of raw materials of petrochemical origin with raw materials of renewable origin in polymer production is a priority research area.

The literature to date describes the use of various natural polymers or polymers from renewable raw materials, the monomer units of which come from the family of sugars (glucose, arabinose, xylose, galactose, mannose, ribose, glucuronic acid, etc.) or from the family of amino acids (glutamic acid, aspartic acid, lysine, etc.). These polymers are predominantly linear or branched, depending on the plant from which they are obtained or according to their manufacturing process.

An example of a polymer of natural origin is polyglutamic acid (PGA), which is currently the subject of numerous research studies. It is a predominantly linear polymer and consists of glutamic acid (GA) monomer units. Glutamic acid is an amino acid characterized by an amine function in the α-position and by two carboxylic acid functions (or carboxylates depending on the pH) in the α and γ positions (cf. chemical formula 1).

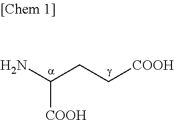

Chemical structure of glutamic acid (GA).

One of the ways to increase branching in a synthetic or natural polymer or polymer of natural origin is to carry out crosslinking reactions. The purpose of crosslinking polymer chains is to link together a plurality of polymer chains, which, when added to a polar phase, and more particularly to water, take the form of a three-dimensional network that although insoluble in water is swellable with water, resulting in an aqueous gel.

Crosslinked polymers may be prepared:

In one step by reacting the monomers and the crosslinking agent during the polymerization reaction, or

In at least two steps, the first of which consists of producing the polymer, and the second consists of reacting the polymer with a crosslinking agent to obtain a crosslinked polymer.

There are various reactions for crosslinking PGA that make it possible to obtain polymers of natural origin having improved thickening properties in polar media, and especially in aqueous media.

Of the crosslinking agents known to be used in the PGA crosslinking reaction, polyepoxide derivatives are the most widely described since they make it possible to carry out crosslinking processes under environmentally friendly conditions (moderate temperatures, reactions in aqueous media and in the absence of harmful solvents).

However, the implementation of these processes involves diluting the PGA to high levels, which results in a composition in the form of an aqueous gel containing, per 100% of its weight, a content of less than or equal to 10% by weight of a polymer (P), which is difficult for formulators to implement.

A problem that arises therefrom is that of providing a user-friendly composition comprising polymers that are of natural origin, the raw materials of which are renewable, and that have thickening properties in polar media and more particularly in aqueous media.

A solution provided by the present invention is a composition (CA) in the form of a self-inverting water-in-oil emulsion containing, per 100% of its weight, a content of greater than or equal to 20% by weight of a polymer (P) consisting of monomer units derived from partially or totally salified glutamic acid (GA) and of units derived from at least one crosslinking agent (XLA) bearing at least two glycidyl functions.

For the purposes of the present invention, what is meant by a “water-in-oil emulsion” is a heterogeneous mixture of two immiscible liquids, one being dispersed in the form of small droplets in the other, said mixture being thermodynamically unstable and stabilized by the presence of a surfactant system comprising at least one emulsifying surfactant.

For the purposes of the present invention, what is meant by a “self-inverting water-in-oil emulsion” is a water-in-oil emulsion as defined above in which the emulsifying surfactants present give the emulsion a hydrophilic-lipophilic balance (HLB) such that, once said emulsion has been added to a polar phase, for example water, the direction of the emulsion will change from water-in-oil to oil-in-water, thereby bringing the polymer (P) into contact with the polar phase to be thickened.

In the polymer (P) present in the composition (CA) that is the subject of the present invention, the monomer units derived from partially or totally salified glutamic acid (GA) are linked together:
either in such a way that the amine function of a glutamic acid (GA) monomer unit is covalently linked to the carboxylic function present in the alpha (α) position of a second glutamic acid (GA) monomer unit; the resulting polymer is then referred to as a “α-polyglutamic acid” or PAGA (cf. chemical formula 2), which is partially or totally salified,

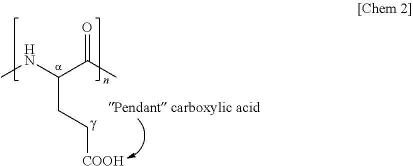

Chemical structure of α-polyglutamic acid or PAGA.
or in such a way that the amine function of a glutamic acid (GA) monomer unit is covalently linked to the carboxyl function of the side chain present in the gamma (γ) position of a second glutamic acid (GA) monomer unit; the resulting polymer is then referred to as a “γ-polyglutamic acid” or PGGA (cf. chemical formula 3), which is partially or totally salified.

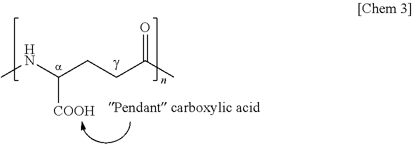

Chemical structure of γ-polyglutamic acid or PGGA.

In general, PGA can be prepared chemically according to peptide synthesis methods known to those skilled in the art, especially through sequential steps of selective protection, activation, coupling, and deprotection. The coupling generally consists of a nucleophilic attack of the amine function of a glutamic acid monomer unit at an activated carboxylic acid function of another glutamic acid monomer unit.

PGGA can also be obtained by processes comprising at least one microbial fermentation step involving the use of at least one bacterial strain.

For the purposes of the present invention, in the polymer (P) as defined previously the term “salified” indicates that the “pendant” carboxylic acid function present on each glutamic acid (GA) monomer unit of the polymer (in the gamma position in the case of PAGA or in the alpha position in the case of PGGA) is present in an anionic or carboxylate form. The counterion of this carboxylate function is a cation derived for example from salts of alkali metals such as sodium or potassium or salts of nitrogen bases such as amines, lysine or monoethanolamine (HO—CH2—CH2—NH2).

For the purposes of the present invention, what is meant by a “crosslinking agent (XLA)” is a chemical substance having a structure that allows it to link covalently to at least two polymer chains.

For the purposes of the present invention, what is meant by a “crosslinking agent (XLA) bearing at least two glycidyl functions” is a crosslinking agent (XLA) as defined above in which the molecular structure contains at least two glycidyl units or functions of the formula (I′):

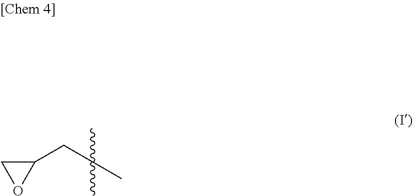 [SMILES: *CC1CO1]

The crosslinking of the polymer chains of the polymer (P) takes place by a reaction between the terminal free amine function (—NH2) and/or one or more “pendant” or terminal carboxyl or carboxylate functions (—COOH or —COO—) present in the structure of said polymer (P) and at least one epoxy group present in the structure of the crosslinking agent (XLA) bearing at least two glycidyl functions.

Depending on the particular case, the composition (CA) of the invention may exhibit one or more of the following features:
the content by weight of the polymer (P) is greater than or equal to 20% and less than or equal to 60%;
the polymer (P) is gamma-polyglutamic acid (PGGA) in acid form or in partially or totally salified form;
in the polymer (P), per 100 mol % of monomer units derived from partially or totally salified glutamic acid (GA), the crosslinking agent (XLA) represents from 0.5 mol % to 20 mol %;
the composition (CA) has a viscosity of between 100 mPa·s and 10 000 mPa·s (measured with a Brookfield RVT viscometer, speed 5 rpm);
the composition (CA) also comprises a monomer unit derived from the compound of the formula (X′):

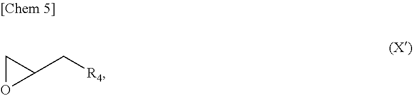

where R4 represents a linear or branched, saturated or unsaturated, functionalized or non-functionalized hydrocarbon radical containing from 6 to 22 carbon atoms.

According to a particular aspect, R4 represents a hydrocarbon radical selected from the group consisting of the heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl, and dodecosyl radicals.

According to another particular aspect, in said polymer (P), per 100% of the weight of monomer units derived from partially or totally salified glutamic acid (GA), the monomer units derived from the compound of the formula (X′) represent from 1% to 50% by weight.

In addition, the crosslinking agent (XLA) may be selected from the group consisting of:
ethylene glycol diglycidyl ether of the formula (I)

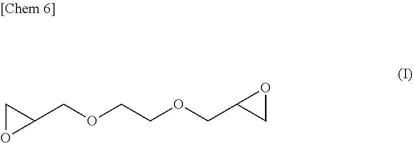 [SMILES: C(COCC1CO1)OCC1CO1]

the compound of the formula (II)

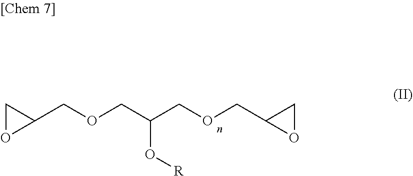

where R represents a hydrogen atom or the glycidyl radical

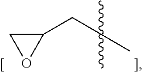 [SMILES: *CC1CO1]

and n represents an integer greater than or equal to one and less than or equal to 10.

When R represents a hydrogen atom and n is equal to 1, the compound of the formula (II) is more particularly the compound of the formula (IIa), or glycerol diglycidyl ether.

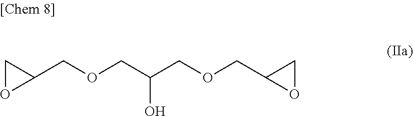 [SMILES: OC(COCC1CO1)COCC1CO1]

When R represents

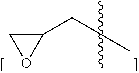 [SMILES: *CC1CO1]

and n is equal to 1, the compound of the formula (II) is more particularly the compound of the formula (IIb), or glycerol triglycidyl ether.

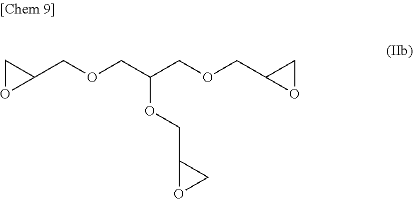 [SMILES: C(OCC1CO1)C(COCC1CO1)OCC1CO1]

When R represents a hydrogen atom and n is equal to 2, the compound of the formula (II) is more particularly the compound of the formula (IIc), or diglycerol diglycidyl ether.

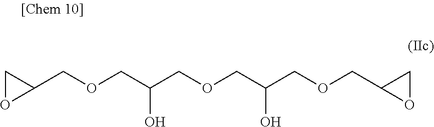 [SMILES: OC(COCC(O)COCC1CO1)COCC1CO1]

When R represents

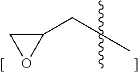 [SMILES: *CC1CO1]

and n is equal to 2, the compound of the formula (II) is more particularly the compound of the formula (IId), or diglyceryl tetraglycidyl ether.

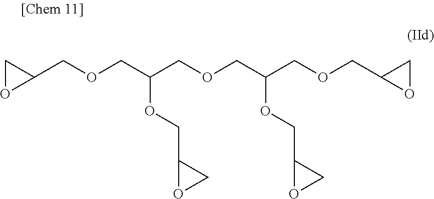 [SMILES: C(OCC(COCC1CO1)OCC1CO1)C(COCC1CO1)OCC1CO1]

propane-1,3-diol diglycidyl ether of the formula (III)

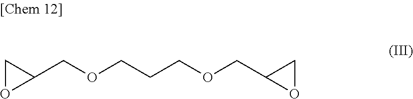 [SMILES: C(COCC1CO1)COCC1CO1]

propane-1,2-diol diglycidyl ether of the formula (IV)

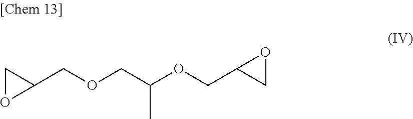

butane-1,4-diol diglycidyl ether of the formula (V)

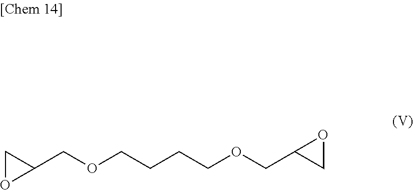 [SMILES: C(CCOCC1CO1)COCC1CO1]

butane-1,2-diol diglycidyl ether of the formula (VI)

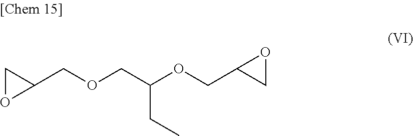

butane-1,3-diol diglycidyl ether of the formula (VII)

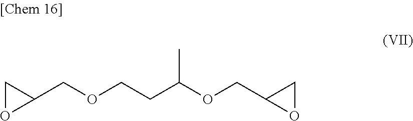

hexane-1,6-diol diglycidyl ether of the formula (VIII)

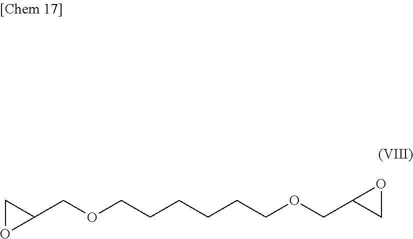 [SMILES: C(CCCOCC1CO1)CCOCC1CO1]

the compound of the formula (IX)

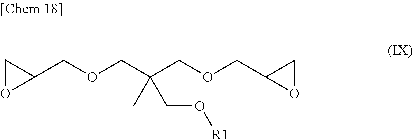

where R1 represents a hydrogen atom or the glycidyl radical

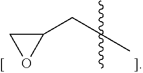 [SMILES: *CC1CO1]

When R1 represents a hydrogen atom, the compound of the formula (IX) is more particularly the compound of the formula (IXa), or trimethylolethane diglycidyl ether.

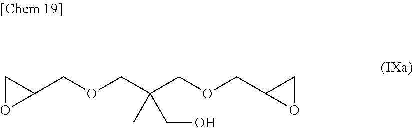 [SMILES: CC(CO)(COCC1CO1)COCC1CO1]

When R1 represents the glycidyl radical

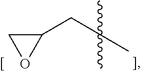 [SMILES: *CC1CO1]

the compound of the formula (IX) is more particularly the compound of the formula (IXb), or trimethylolethane triglycidyl ether.

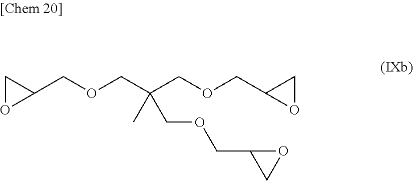 [SMILES: CC(COCC1CO1)(COCC1CO1)COCC1CO1]

the compound of the formula (X)

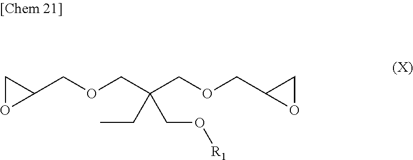

where R1 represents a hydrogen atom or the glycidyl radical

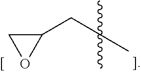 [SMILES: *CC1CO1]

When R1 represents a hydrogen atom, the compound of the formula (X) is more particularly the compound of the formula (Xa), or trimethylolpropane diglycidyl ether.

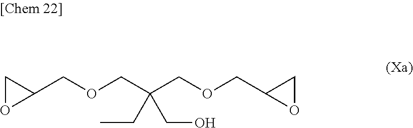 [SMILES: CCC(CO)(COCC1CO1)COCC1CO1]

When R1 represents the glycidyl radical

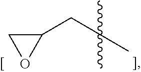 [SMILES: *CC1CO1]

the compound of the formula (X) is more particularly the compound of the formula (Xb), or trimethylolpropane triglycidyl ether.

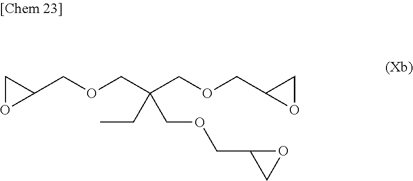 [SMILES: CCC(COCC1CO1)(COCC1CO1)COCC1CO1]

the compound of the formula (XI)

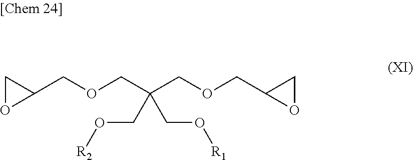

where R1 and R2 each independently represent a hydrogen atom or the glycidyl radical

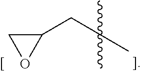 [SMILES: *CC1CO1]

When R1 and R2 each represent a hydrogen atom, the compound of the formula (XI) is more particularly the compound of the formula (XIa), or pentaerythritol diglycidyl ether.

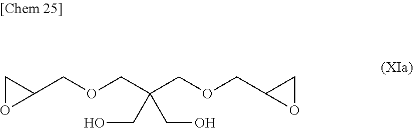 [SMILES: OCC(CO)(COCC1CO1)COCC1CO1]

When R1 represents a hydrogen atom and R2 represents the glycidyl radical

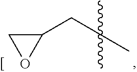 [SMILES: *CC1CO1]

the compound of the formula (XI) is more particularly the compound of the formula (XIb), or pentaerythritol triglycidyl ether.

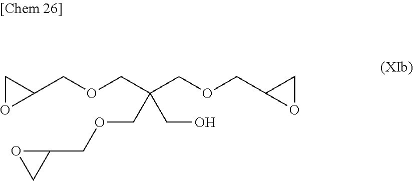 [SMILES: OCC(COCC1CO1)(COCC1CO1)COCC1CO1]

When R1 and R2 each represent the glycidyl radical

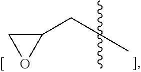 [SMILES: *CC1CO1]

the compound of the formula (XI) is more particularly the compound of the formula (XIc), or pentaerythritol tetraglycidyl ether.

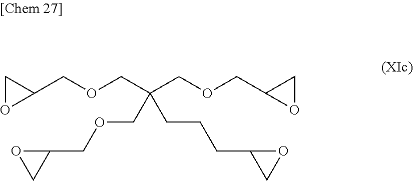 [SMILES: C(CC1CO1)CC(COCC1CO1)(COCC1CO1)COCC1CO1]

the compound of the formula (XII)

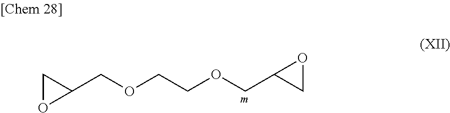

where m represents an integer greater than or equal to 2
the compound of the formula (XIII)

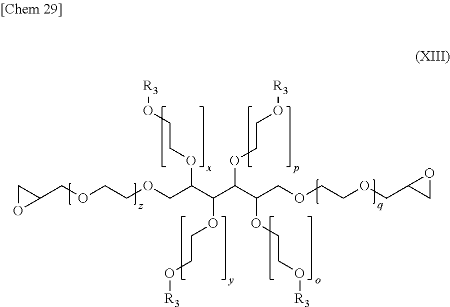

where R3 represents a hydrogen atom or the glycidyl radical

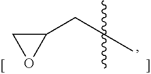 [SMILES: *CC1CO1]

and x, y, z, o, p, and q each independently represent an integer greater than or equal to 2 and less than or equal to 10.

The present invention also provides a process for preparing a pharmaceutical composition (CA) as defined previously, comprising:
a) a step of preparing an aqueous solution comprising partially or totally salified polyglutamic acid (PGA), wherein said aqueous solution comprises, per 100% of its weight, between 5% and 70% by weight of partially or totally salified PGA and a crosslinking agent (XLA) containing at least two glycidyl functions,
b) a step of adjusting the pH of the aqueous solution obtained in step a) to a pH of between 3 and 11,
c) a step of preparing an organic phase comprising at least one volatile oil, at least one other oil that is nonvolatile (H), and at least one water-in-oil emulsifying surfactant (S1),
d) a step of pre-emulsifying by adding the organic phase obtained in step c) to the aqueous solution obtained in step b) with stirring,
e) a step of emulsifying the pre-emulsion obtained in step d) by homogenizing with stirring,
f) a step of distilling the water and volatile oil contained in the emulsion obtained in step e);
g) a step of adding at least one oil-in-water emulsifying surfactant (S2) so as to obtain the composition (CA).

Depending on the particular case, the process of the invention may include one or more of the following features:
in step a) the polymer (P) used is PGGA,
in step a) all of the monomer units making up the PGGA are derived from sodium glutamate, potassium glutamate, ammonium glutamate, calcium glutamate, magnesium glutamate or a mixture of these forms.
in step a) the crosslinking agent (XLA) is present in weight proportions of between 0.5% and 10% by weight relative to the weight of polyglutamic acid (PGA).
in step c) the at least one water-in-oil emulsifying agent (S1) is selected from the group consisting of sorbitan esters, polyglycerol esters, alkoxylated polyglycerol esters, polyglycol polyhydroxystearates, polyglyceryl polyhydroxystearates, and alkoxylated polyglyceryl polyhydroxystearates.
in step c) the organic solution contains, per 100% of its own weight, between 10% and 30% by weight of at least one water-in-oil emulsifying agent (S1), preferably between 15% and 20% by weight.
in step c) the water-in-oil emulsifying agent (S1) is a polyglyceryl polyhydroxystearate.
in step c) the organic solution comprises at least one polymeric surfactant, such as polyesters having a molecular weight of between 1000 and 3000 that are products of the condensation between a polyisobutenyl succinic acid or the anhydride thereof and aminodiethanol, or lastly block copolymers having a molecular weight of between 2500 and 3500. For stabilization of the emulsion produced in step e), this compound is introduced into the fatty phase in a content of 5% to 30% by weight and preferably between 15% and 20%.
in step g) the at least one oil-in-water emulsifying surfactant (S2) is selected from the group consisting of a polyethoxylated fatty alcohol, a polyethoxylated hexitan ester, an alkyl polyglycoside, a composition of alkyl polyglycoside and fatty alcohols, a polyglycerol ester, and a composition of polyglycerol ester and polyglycerol.
step d) is carried out such that the weight ratio between the aqueous solution and the organic phase is between 90/10 and 10/90, preferably between 20/80 and 40/60;
in step a) the aqueous solution also comprises at least one compound of the formula (X′):

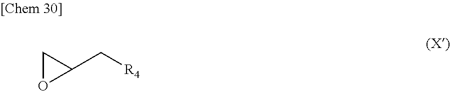

where R4 represents a linear or branched, saturated or unsaturated, functionalized or non-functionalized hydrocarbon radical containing from 6 to 22 carbon atoms.

According to a particular aspect, R4 represents a hydrocarbon radical selected from the group consisting of the heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl, and dodecosyl radicals.
the content of the compound of the formula (X′) in the polar solution is, per 100% by weight of said aqueous solution, between 0.05% and 35% by weight, it being understood that the sum of the weight proportions of the polymer (P), of the crosslinking agent (XLA), of the water, and of the compound of the formula (X′) is equal to 100%.
in step e) the homogenization is carried out with mechanical shear stirring;
in step f) the distillation is carried out under reduced pressure and with heating. This results in crosslinking of the polyglutamic acid and concentration of the emulsion;
in steps c) and f) the volatile oil is a light isoparaffin containing 8 to 11 carbon atoms. This isoparaffin may be selected from those sold under the names Isopar™ G, Isopar™ L, Isopar™ H, and Isopar™ J.

Opting for a concentrated inverse emulsion process makes it possible to dissolve the starting PGGA, its possible co-constituents, and also the crosslinking agent(s) in the aqueous phase of the emulsion. The production of the emulsion makes it possible to create droplets isolated from one other, enabling the crosslinking of the PGA without solidification of the reaction medium due to the increase in viscosity of the aqueous phase during the crosslinking step. The step of concentrating a light fatty phase by distillation affords a product in liquid form that has an active substance content of greater than 20%.

The present invention also provides:
for the use of said composition (CA) of the invention as a thickening and/or emulsifying and/or stabilizing agent for a composition for topical cosmetic use; and
a cosmetic composition for topical use (F) characterized in that it contains as thickening agent, per 100% of its total weight, between 0.1% by weight and 10% by weight of said composition (CA) of the invention.

According to a particular aspect, in the composition (CA) that is the subject of the present invention, the content by weight of the polymer (P) is greater than or equal to 20% and less than or equal to 60%; and more particularly greater than or equal to 20% and less than or equal to 40%.

PGGA can exist in different conformational forms in solution in water. These forms depend on the inter- and intramolecular hydrogen bonds and thus on the pH, the polymer concentration, the ionic strength of the solution, and also the temperature. The PGGA chains can thus take the form of an α-helix, a β-sheet, aggregates, or else be in a disordered and random state.

According to a particular aspect, in the composition (CA) that is the subject of the present invention the polymer (P) is in a helical conformation when it is present in a solution at a content by weight of less than or equal to 0.1% and where said solution has a pH of less than or equal to 7.

According to a particular aspect, in the composition (CA) that is the subject of the present invention the polymer (P) is in a sheet conformation when it is present in a solution at a content by weight of less than or equal to 0.1% and where said solution has a pH above 7.

According to a particular aspect of the composition (CA) that is the subject of the present invention, in the polymer (P), per 100 mol % of monomer units derived from partially or totally salified glutamic acid (GA), the crosslinking agent (XLA) represents from 1 mol % to 20 mol %, and even more particularly from 1 mol % to 18 mol %.

According to another particular aspect, the composition (CA) has a viscosity of between 1000 mPa·s and 10 000 mPa·s (measured with a Brookfield RVT viscometer, speed 5 rpm), more particularly between 1000 mPa·s and 5000 mPa·s.

According to another particular aspect, the crosslinking agent (XLA) is ethylene glycol diglycidyl ether of the formula (I).

According to another particular aspect, in step a) of the process that is the subject of the present invention the partially or totally salified polyglutamic acid (PGA) is in the form of a potassium, sodium or ammonium salt, and more particularly in the form of a sodium salt.

According to another particular aspect, in step a) of the process that is the subject of the present invention the aqueous solution contains, per 100% of its weight, between 5% and 60% by weight, more particularly between 10% and 50% by weight, of partially or totally salified polyglutamic acid (PGA).

According to another particular aspect, in step a) of the process that is the subject of the present invention the crosslinking agent (XLA) is selected from at least one of the group consisting of the compounds of the formulas (I), (II), (III), (IV), (V), (VI), (VII), (VIII), (IX), (X), (XI), (XII), and (XIII) as defined previously.

According to another aspect, in step c) of the process that is the subject of the present invention what is meant by a “volatile oil” is a fatty substance that is liquid at a temperature of 25° C. and at atmospheric pressure and has a flash point of between 40 and 100° C.

According to a more particular aspect, for the purposes of the present invention what is meant by a “volatile oil” is one of the group consisting of branched alkanes containing from seven to 40 carbon atoms, such as isododecane, isopentadecane, isohexadecane, isoheptadecane, isooctadecane, isononadecane or isoeicosane, or mixtures of some of these such as those mentioned below and identified by their INCI name: C7-8 Isoparaffin, C8-9 Isoparaffin, C9-11 Isoparaffin, C9-12 Isoparaffin, C9-13 Isoparaffin, C9-14 Isoparaffin, C9-16 Isoparaffin, C10-11 Isoparaffin, C10-12 Isoparaffin, C10-13 Isoparaffin, C11-12 Isoparaffin, C11-13 Isoparaffin, and C11-14 Isoparaffin.

According to an even more particular aspect, for the purposes of the present invention what is meant by a “volatile oil” is at least one of the group consisting of isododecane, isohexadecane, C7-8 Isoparaffin, C8-9 Isoparaffin, C9-11 Isoparaffin, C11-13 Isoparaffin, and C11-14 Isoparaffin.

According to another even more particular aspect of the present invention, the volatile oil is selected from the group consisting of C8-9 Isoparaffin, C9-11 Isoparaffin, C11-13 Isoparaffin, and C11-14 Isoparaffin.

According to another even more particular aspect of the present invention, the “volatile oil” is selected from the group consisting of the isoparaffins sold under the brand names Isopar™ G, Isopar™ L, Isopar™ H and Isopar™ J.

According to another aspect, in step c) of the process that is the subject of the present invention what is meant by “oil (O)” is a fatty substance that is liquid at a temperature of 25° C. at atmospheric pressure, especially:
linear alkanes containing from 11 to 19 carbon atoms;
branched alkanes containing from 11 to 40 carbon atoms, such as isododecane, isopentadecane, isohexadecane, isoheptadecane, isooctadecane, isononadecane or isoeicosane, or mixtures of some of these such as those mentioned below and identified by their INCI name: C12-14 Isoparaffin, C12-20 Isoparaffin, C13-14 Isoparaffin, and C13-16 Isoparaffin;
cycloalkanes optionally substituted with one or more linear or branched alkyl radicals;
white mineral oils, such as those sold under the following names: Marcol™ 52, Marcol™ 82, Drakeol™ 6VR, Eolane™ 130, and Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes containing from 15 to 19 carbon atoms, said alkanes being linear alkanes, branched alkanes, and cycloalkanes, and more particularly the mixture (M1) that contains, per 100% of its weight:
a weight proportion of branched alkanes of greater than or equal to 90% and less than or equal to 100%,
a weight proportion of linear alkanes of greater than or equal to 0% and less than or equal to 9%,
a weight proportion of cycloalkanes of greater than or equal to 0% and less than or equal to 1%, and
   said mixture (M1) being characterized in that it contains, per 100% of its weight:
a weight proportion of greater than or equal to 95% of branched alkanes, linear alkanes, and cycloalkanes and less than or equal to 100% containing from 15 to 19 carbon atoms, and
a weight proportion of greater than or equal to 0% and less than or equal to 5% of branched alkanes, linear alkanes, and cycloalkanes containing fewer than 14 carbon atoms and of linear alkanes and cycloalkanes containing more than 20 carbon atoms.

For the purposes of the present invention, what is meant by “linear alkanes” present in the mixture (M1) as defined above and containing from 15 to 19 carbon atoms is more particularly ones selected from the group consisting of n-pentadecane, n-hexadecane, n-heptadecane, n-octadecane, and n-nonadecane.

For the purposes of the present invention, what is meant by “branched alkanes” present in the mixture (M1) as defined above and containing from 15 to 19 carbon atoms is more particularly ones selected from the group consisting of isopentadecane, isohexadecane, isoheptadecane, isooctadecane, and isononadecane.

The mixture (M1) is more particularly the mixture sold under the brand name Emogreen™ L15 or else the mixture sold under the brand name Emogreen™ L19.
fatty alcohol ethers of the formula (XIV):

Z1-O—Z2 (XIV),

in which Z1 and Z2, which may be identical or different, represent a linear or branched alkyl radical containing from five to 18 carbon atoms, for example dioctyl ether, didecyl ether, didodecyl ether, dodecyl octyl ether, dihexadecyl ether, 1,3-dimethylbutyl tetradecyl ether, 1,3-dimethylbutyl hexadecyl ether, bis(1,3-dimethylbutyl) ether or dihexyl ether.
monoesters of fatty acids and alcohols of the formula (XV):

R′1-(C═O)—O—R′2 (XV),

in which R′1-(C═O) represents a saturated or unsaturated, linear or branched acyl radical containing from eight to 24 carbon atoms, and R′2 represents, independently of R′1, a saturated or unsaturated, linear or branched hydrocarbon chain containing from one to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, or isostearyl isostearate;
diesters of fatty acids and glycerol of the formulas (XVI) and (XVII):

R′3-(C═O)—O—CH2—CH(OH)—CH2—O—(C═O)—R′4 (XVI)

R′5-(C═O)—O—CH2—CH[O—(C═O)—R′6]—CH2—OH (XVII),

in which R′3-(C═O) and R′4-(C═O), R′5-(C═O), R′6-(C═O), which may be identical or different, represent a saturated or unsaturated, linear or branched acyl group containing from eight to 24 carbon atoms.
triesters of fatty acids and glycerol of the formula (XVIII):

R′7-(C═O)—O—CH2—CH[O—(C═O)-R″8]—CH2—O—(C═O)-R″9 (XVIII),

in which R′7-(C═O), R′8-(C=0) and R′9-(C═O), which may be identical or different, represent a saturated or unsaturated, linear or branched acyl group containing from eight to 24 carbon atoms.

According to another particular aspect of the present invention, said oil (H) is selected from:
undecane, tridecane, isododecane or isohexadecane;
mixtures of alkanes and isoalkanes and cycloalkanes such as the mixture (M1) as defined previously and the mixtures sold under the names
Emogreen™ L15, Emogreen™ L19, Emosmart™ L15, Emosmart™ L19, Emosmart™ V21, Isopar™ M;
white mineral oils sold under the names Marcol™ 52, Marcol™ 82,
Drakeol™ 6VR, Eolane™ 130 or Eolane™ 150;
hemisqualane, squalane, hydrogenated polyisobutene or hydrogenated polydecene;
dioctyl ether or didecyl ether;
isopropyl myristate, hexyl palmitate, octyl palmitate, isostearyl isostearate, octanoyl/decanoyl triglyceride, hexadecanoyl/octadecanoyl triglyceride, and triglycerides derived from rapeseed oil, sunflower oil, linseed oil or palm oil.

According to another aspect, in step c) of the process that is the subject of the present invention what is meant by “water-in-oil emulsifying surfactant (S1)” is an emulsifying surfactant having an HLB (hydrophilic-lipophilic balance) value low enough to induce the formation of a water-in-oil emulsion, namely an emulsion in which the aqueous phase will be dispersed and stabilized in the oily organic phase.

Examples of a water-in-oil emulsifying surfactant include anhydrohexitol esters of linear or branched, saturated or unsaturated aliphatic carboxylic acids containing from 12 to 22 carbon atoms, optionally substituted with one or more hydroxyl groups, and more particularly esters of anhydrohexitols selected from anhydrosorbitols and anhydromannitols and of linear or branched, saturated or unsaturated aliphatic carboxylic acids containing from 12 to 22 carbon atoms, optionally substituted with one or more hydroxyl groups.

In step c) of the process that is the subject of the present invention the water-in-oil emulsifying system (S1) is more particularly selected from the group consisting of
sorbitan laurate, for example the product sold under the name Montane™ 20,
sorbitan palmitate, for example the product sold under the name Montane™ 40,
sorbitan stearate, for example the product sold under the name Montane™ 60,
sorbitan oleate, for example the product sold under the name Montane™ 80,
sorbitan sesquioleate, for example the product sold under the name Montane™ 83,
sorbitan trioleate, for example the product sold under the name Montane™ 85,
sorbitan isolaurate,
sorbitan isostearate, for example the product sold under the name Montane™ 70,
mannitan laurate, mannitan oleate, or a mixture of these esters; polyesters having a molecular weight of between 1000 and 3000 g/mol and resulting from the condensation between a polyisobutenyl succinic acid or the anhydride thereof, such as Hypermer™ 2296, or the mixture sold under the brand name Simaline™ IE 501 A.

Examples of a water-in-oil emulsifying surfactant (S1) include the polyglycerol esters represented by a compound of the formula (XIX):

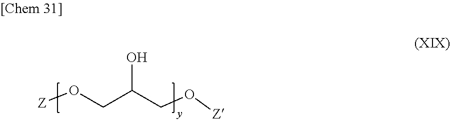

in which Z represents an acyl radical of formula R2-C(═O)— in which R2 represents a saturated or unsaturated, linear or branched aliphatic hydrocarbon radical containing from 11 to 35 carbon atoms and more particularly a radical selected from the dodecanoyl, tetradecanoyl, hexadecanoyl, octadecanoyl, eicosanoyl, docosanoyl, oleyl, linoleyl, linolenoyl or isostearyl radicals, Z′ represents the acyl radical of formula R2-C(═O)— as defined above, where Z′ is identical to or different from Z, or a hydrogen atom, and y represents an integer greater than or equal to 2 and less than or equal to 20.

According to a more particular aspect, the compound of the formula (XIX) is selected from the group consisting of decaglyceryl oleate, decaglyceryl isostearate, decaglyceryl monolaurate, decaglyceryl monolinoleate, and decaglyceryl monomyristate.

Examples of a water-in-oil emulsifying surfactant (S1) include the alkoxylated polyglycerol esters represented by a compound of the formula (XX):

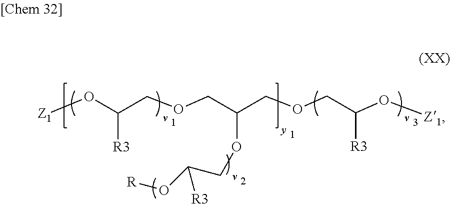

in which Z1 represents an acyl radical of formula R′2-C(═O)— in which R′2 represents a saturated or unsaturated, linear or branched aliphatic hydrocarbon radical containing from 11 to 35 carbon atoms, and more particularly a radical selected from the dodecanoyl, tetradecanoyl, hexadecanoyl, octadecanoyl, eicosanoyl, docosanoyl, oleyl, linoleyl, linolenoyl or isostearyl radicals, Z1′ represents the acyl radical of formula R′2-C(═O)— as defined above, where Z1′ is identical to or different from Z1, or a hydrogen atom, R3 represents a hydrogen atom, a methyl radical, or an ethyl radical, y1 represents an integer greater than or equal to 2 and less or equal to 20, v1, v2, v3 are identical or different and represent an integer greater than or equal to 0 and less than or equal to 50, and the sum [(y1.v1)+(y1.v2)+v3)] is an integer greater than or equal to 1 and less than or equal to 50.

Examples of a water-in-oil emulsifying surfactant (S1) include the polyglycol polyhydroxystearates of the formula (XXI):

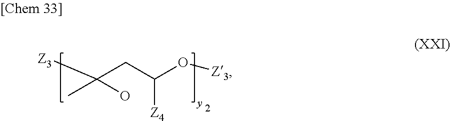

formula (XXI) in which y2 represents an integer greater than or equal to 2 and less than or equal to 50, Z4 represents a hydrogen atom, a methyl radical or an ethyl radical, and Z3 represents a radical of the formula (XXII):

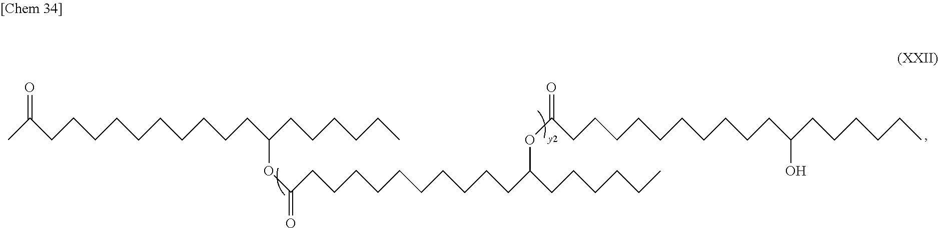

formula (XXII) in which y′2 represents an integer greater than or equal to 0 and less than or equal to 10, more particularly greater than or equal to 1 and less than or equal to 10, and Z′3 represents a radical of the formula (XXII) as defined above, where Z3′ is identical to or different from Z3, or a hydrogen atom.

Examples of a water-in-oil emulsifying surfactant of the formula (XXI) that may be used for preparing the emulsifying (S1) system include the PEG-30 dipolyhydroxystearate sold under the name Simaline™ WO, or else the mixtures comprising PEG-30 dipolyhydroxystearate and sold under the names Simaline™ IE 201 A and Simaline™ IE 201 B, or else the mixture comprising trimethylolpropane-30 tripolyhydroxystearate sold under the name Simaline™ IE 301 B.

Examples of a water-in-oil emulsifying surfactant (S1) include the polyglyceryl polyhydroxystearates represented by formula (XXIII):

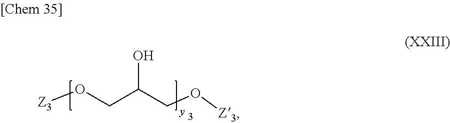

in which Z3 represents a radical of the formula (XXIII) as defined above and Z′3 represents a radical of the formula (XXII) as defined above, where Z3′ is identical to or different from Z3, or a hydrogen atom, and y3 represents an integer greater than or equal to 2 and less than or equal to 20.

Examples of a water-in-oil emulsifying surfactant (S1) include the alkoxylated polyglyceryl polyhydroxystearate compounds represented by formula (XXIV):

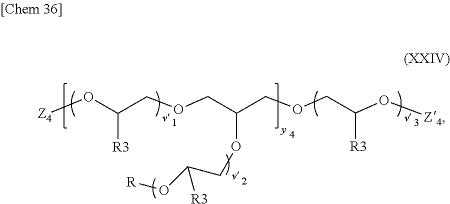

in which Z4 represents a radical of the formula (XXII) as defined above and Z′4 represents a radical of the formula (XXII) as defined above, where Z4′ is identical to or different from Z4, or a hydrogen atom, y4 represents an integer greater than or equal to 2 and less than or equal to 20, v′1, v′2, v′3, which may be identical or different, represent an integer greater than or equal to 0 and less than or equal to 50, and the sum [(y4.v′1)+(y4.v′2)+v′3)] is an integer greater than or equal to 1 and less than or equal to 50.

According to another aspect, in step g) of the process that is the subject of the present invention what is meant by “oil-in-water emulsifying surfactant (S2)” is an emulsifying surfactant having an HLB value high enough to induce the formation of an oil-in-water emulsion, namely an emulsion in which the oily organic phase will be dispersed and stabilized in the aqueous phase.

According to another aspect, in step g) of the process that is the subject of the present invention the oil-in-water surfactant (S2) may be the “polyethoxylated fatty alcohols” denoted by the compounds of the formula (XXV):

R″—O—(CH2—CH2—O)n′—OH (XXV),

where R″ represents a linear or branched, saturated or unsaturated hydrocarbon radical that may bear hydroxyl groups and contains from six to 22 carbon atoms, and where n′ represents an integer greater than or equal to four and less than or equal to one hundred.

According to a more particular aspect, in formula (XXV) R″ represents a linear or branched, saturated hydrocarbon radical containing from ten to 22 carbon atoms.

According to an even more particular aspect, the compound of the formula (XXV) is a linear decyl alcohol ethoxylated with six moles of ethylene oxide, a linear decyl alcohol ethoxylated with eight moles of ethylene oxide, a linear lauryl alcohol ethoxylated with six moles of ethylene oxide, a linear lauryl alcohol ethoxylated with seven moles of ethylene oxide, a linear lauryl alcohol ethoxylated with eight moles of ethylene oxide, a linear tridecyl alcohol ethoxylated with six moles of ethylene oxide, a linear tridecyl alcohol ethoxylated with eight moles of ethylene oxide or a linear tridecyl alcohol ethoxylated with nine moles of ethylene oxide.

According to another aspect, in step g) of the process that is the subject of the present invention the oil-in-water surfactant (S2) may be polyethoxylated hexitan esters, and in particular polyethoxylated sorbitan esters, in which the aliphatic hydrocarbon chain contains from 12 to 22 carbon atoms and in which the number of ethylene oxide units is between 5 and 40, for example the sorbitan oleate ethoxylated with 20 mol of ethylene oxide sold under the trade name Montanox™ 80 or the sorbitan laurate ethoxylated with 20 mol of ethylene oxide sold under the trade name Montanox™ 20.

According to another aspect, in step g) of the process that is the subject of the present invention the oil-in-water surfactant (S2) may be the alkyl polyglycosides compositions (C1) represented by formula (XXVI):

R″1-O-(G)x-H (XXVI)

in which x, or the average degree of polymerization, represents a decimal number between 1.05 and 5, G represents a reducing sugar residue, and R″1 represents a saturated or unsaturated, linear or branched aliphatic hydrocarbon radical optionally substituted with one or more hydroxyl groups and containing from 12 to 36 carbon atoms, said composition (C1) consisting of a mixture of compounds represented by the formulas (XXVI1), (XXVI2), (XXVI3), (XXVI4), and (XXVI5):

R″1-O-(G)1-H (XXVI1)

R″1-O-(G)2-H (XXVI2)

R″1-O-(G)3-H (XXVI3)

R″1-O-(G)4-H (XXVI4)

R″1-O-(G)5-H (XXVI5)

in the respective molar proportions a1, a2, a3, a4, and a5 such that:
the sum a1+a2+a3+a4+a5 is equal to 1 and that
the sum a1+2a2+3a3+4a4+5a5 is equal to x.

What is meant by “saturated or unsaturated, linear or branched aliphatic hydrocarbon radical containing from 12 to 36 carbon atoms, optionally substituted with one or more hydroxyl groups” is, for the radical R″1 in formula (XXVI) as defined above, more particularly the n-dodecyl radical, the n-tetradecyl radical, the n-hexadecyl radical, the n-octadecyl radical, the n-eicosyl radical, the n-docosyl radical or the 12-hydroxyoctadecyl radical.

What is meant by “reducing sugar” in the definition of the formula (XXVI) as defined above is saccharide derivatives that do not have in their structures any glycoside bond between an anomeric carbon and the oxygen of an acetal group as defined in the reference publication: “Biochemistry”, Daniel Voet/Judith G. Voet, page 250, John Wiley & Sons, 1990. The oligomeric structure (G)x may be present in any isomeric form, whether this be due to optical isomerism, geometrical isomerism or regioisomerism; it may also represent a mixture of isomers.

In formula (XXVI) as defined above the group R1-O— is linked to G by the anomeric carbon of the saccharide residue, so as to form an acetal function.

According to a particular aspect, G in the definition of the formula (XXVI) as defined above represents a reducing sugar residue selected from glucose, dextrose, sucrose, fructose, idose, gulose, galactose, maltose, isomaltose, maltotriose, lactose, cellobiose, mannose, ribose, xylose, arabinose, lyxose, allose, altrose, dextran, and tallose; more particularly, G represents a reducing sugar residue selected from glucose, xylose, and arabinose residues.

According to an even more particular aspect, x in the definition of the formula (XXVI), or the average degree of polymerization, represents a decimal number greater than or equal to 1.05 and less than or equal to 2.5, more particularly greater than or equal to 1.05 and less than or equal to 2.0, and even more particularly greater than or equal to 1.25 and less than or equal to 2.0.

According to another aspect, in step g) of the process that is the subject of the present invention the oil-in-water surfactant (S2) may be the compositions (C2) containing, per 100% of their weight:
from 10% to 50% by weight, more particularly from 15% to 40% by weight, and even more particularly from 20% to 30% by weight, of at least one composition (C1) represented by formula (XXVI) as defined previously,
from 90% to 50% by weight, more particularly from 85% to 60% by weight, and even more particularly from 80% to 70% by weight, of at least one fatty alcohol of the formula (XXVII):

R′″1-OH (XXVII),
in which R′″1, which may be identical to or different from R″1, represents a saturated or unsaturated, linear or branched aliphatic hydrocarbon radical optionally substituted with one or more hydroxyl groups and containing from 12 to 36 carbon atoms, preferably from 12 to 22 carbon atoms.

Examples of an oil-in-water emulsifying surfactant (S2) include the polyglycerol esters of the formula (XXVIII):

R12-(C═O)[O—CH2—CH(OH)—CH2]p12-OH (XXVIII),

formula (XVIII) in which p12 represents an integer greater than or equal to one and less than or equal to 15 and in which the group R1-(C═O)— represents a saturated or unsaturated, linear or branched aliphatic radical containing from six to 22 carbon atoms.

Examples of an oil-in-water emulsifying surfactant (S2) include the compositions (C13) containing, per 100% of their weight:
from 10% by weight to 60% by weight of at least one compound of the formula (XXIX):

HO—[CH2—CH(OH)—CH2—O] n12-H (XXIX)

formula (I) in which n12 represents an integer greater than or equal to one and less than or equal to 15; and
from 40% by weight to 90% by weight of at least one compound of the formula (XXVIII) as defined previously.

According to a particular aspect, said use of the composition (CA), which is a subject of the present invention, consists of the thickening of polar phases, for example aqueous, alcoholic or aqueous-alcohol phases or polar phases comprising polyols such as glycerol.

According to another particular aspect, said use consists of the stabilization of an oil-in-water or water-in-oil emulsion, giving said emulsion a homogeneous appearance during storage under various conditions, and more particularly at 25° C. for a time at least equal to one month, and more particularly at 4° C. for a time at least equal to one month, and more particularly at 45° C. for a period at least equal to one month.

According to another particular aspect, said use consists of the stabilization of solid particles in topical cosmetic or dermocosmetic compositions. These solid particles to be suspended may have various regular or irregular geometries, and may be in the form of pearls, beads, rods, flakes, strips or polyhedra. These solid particles are characterized by an average apparent diameter of between one micrometer and five millimeters, more particularly between 10 micrometers and one millimeter.

The solid particles that can be suspended and stabilized by the water-in-oil emulsion (CA) as defined previously in cosmetic, dermopharmaceutical or pharmaceutical topical compositions include micas, iron oxide, titanium oxide, zinc oxide, aluminum oxide, talc, silica, kaolin, clays, boron nitride, calcium carbonate, magnesium carbonate, magnesium hydrogen carbonate, inorganic colored pigments, polyamides, such as nylon-6, polyethylenes, polypropylenes, polystyrenes, polyesters, acrylic or methacrylic polymers, such as polymethyl methacrylates, polytetrafluoroethylene, crystalline or microcrystalline waxes, porous spheres, selenium sulfide, zinc pyrithione, starches, alginates, plant fibers, loofah particles, and sponge particles.

According to another aspect, the cosmetic composition for topical use (F) that is the subject of the present invention contains as thickening agent, per 100% of its total weight, between 0.1% by weight and 8% by weight, more particularly between 0.5% and 8% by weight , and even more particularly between 0.5% and 5% by weight, of said composition (CA) of the invention.

The expression “topical” used in the definition of said composition (F) means that it is employed by application to the skin, hair, scalp or mucous membranes, whether this be a direct application, in the case of a cosmetic or dermocosmetic preparation, or an indirect application, for example in the case of a body care product in the form of a textile or paper wipe or of sanitary products intended to be in contact with the skin or the mucous membranes.

Said composition (F) is generally in the form of an aqueous or aqueous-alcohol or aqueous-glycol solution, in the form of a suspension, of an emulsion, of a microemulsion or of a nanoemulsion, whether they be of the water-in-oil, oil-in-water, water-in-oil-in-water or oil-in-water-in-oil type.

Said composition (F) as defined previously may be packaged in a bottle, in a device of the pump-bottle type, in pressurized form in an aerosol device, in a device equipped with an openwork wall such as a grate, or in a device equipped with a ball applicator (roll-on).

In general, said composition (F) also comprises excipients and/or active ingredients typically employed in the field of formulations for topical use, in particular cosmetic or dermocosmetic formulations, such as thickening and/or gelling surfactants, stabilizers, film-forming compounds, hydrotropic agents, plasticizing agents, emulsifying and coemulsifying agents, opacifying agents, pearlizing agents, superfatting agents, sequestering agents, chelating agents, antioxidants, fragrances, preservatives, conditioning agents, whitening agents intended for bleaching body hair and the skin, active ingredients intended to provide a treatment action for the skin or hair, sunscreens, mineral fillers or pigments, and particles providing a visual effect or intended for the encapsulation of active ingredients, exfoliating particles or texturing agents.

Examples of foaming and/or detergent surfactants that may be combined with the water-in-oil emulsion (CA) in said composition (F) as defined previously include foaming and/or detergent surfactants that are anionic, cationic, amphoteric or nonionic.

Anionic foaming and/or detergent surfactants that may be combined with the water-in-oil emulsion (CA) in said composition (F) as defined previously include alkali metal salts, alkaline earth metal salts, ammonium salts, amine salts, amino alcohol salts, alkyl ether sulfate salts, alkyl sulfate salts, alkylamido ether sulfate salts, alkylaryl polyether sulfate salts, monoglyceride sulfate salts, α-olefin sulfonate salts, paraffin sulfonate salts, alkyl phosphate salts, alkyl ether phosphate salts, alkyl sulfonate salts, alkylamide sulfonate salts, alkylaryl sulfonate salts, alkyl carboxylate salts, alkyl sulfosuccinate salts, alkyl ether sulfosuccinate salts, alkylamide sulfosuccinate salts, alkyl sulfoacetate salts, alkyl sarcosinate salts, acyl isethionate salts, salts of N-acyl taurates, salts of acyl lactylates, salts of N-acyl derivatives of amino acids, salts of N-acyl derivatives of peptides, salts of N-acyl derivatives of proteins or salts of N-acyl derivatives of fatty acids.

Amphoteric foaming and/or detergent surfactants that may be combined with the water-in-oil emulsion (CA) in said composition (F) as defined previously include alkyl betaines, alkylamido betaines, sultaines, alkylamidoalkyl sulfobetaines, imidazoline derivatives, phosphobetaines, amphopolyacetates, and amphopropionates.

Cationic foaming and/or detergent surfactants that may be combined with the water-in-oil emulsion (CA) in said composition (F) as defined previously include in particular quaternary ammonium derivatives.

Nonionic foaming and/or detergent surfactants that may be combined with the water-in-oil emulsion (CA) in said composition (F) as defined previously include more particularly alkyl polyglycosides containing a linear or branched, saturated or unsaturated aliphatic radical containing from 8 to 16 carbon atoms, such as octyl polyglucoside, decyl polyglucoside, undecylenyl polyglucoside, dodecyl polyglucoside, tetradecyl polyglucoside, hexadecyl polyglucoside or dodecane-1,12-diyl polyglucoside; ethoxylated hydrogenated castor oil derivatives, such as the product sold under the INCI name “PEG-40 Hydrogenated Castor Oil”; polysorbates, such as Polysorbate 20, Polysorbate 40, Polysorbate 60, Polysorbate 70, Polysorbate 80 or Polysorbate 85, cocamides or N-alkylamines.

Examples of thickening and/or gelling surfactants that may be combined with the water-in-oil emulsion (CA) in said composition (F) as defined previously include optionally alkoxylated alkyl polyglycoside fatty esters, for example ethoxylated methyl polyglucoside esters, such as the PEG 120 methyl glucose trioleate and PEG 120 methyl glucose dioleate sold respectively under the names Glutamate™ LT and Glutamate™ DOE120; alkoxylated fatty esters, such as the PEG 150 pentaerythrityl tetrastearate sold under the name Crothix™ DS53, the PEG 55 propylene glycol oleate sold under the name Antil™ 141; fatty-chain polyalkylene glycol carbamates, such as the PPG-14 laureth isophoryl dicarbamate sold under the name Elfacos™ T211, and the PPG-14 palmeth-60 hexyl dicarbamate sold under the name Elfacos™ GT2125.

Examples of thickeners and/or gelling agents that may be combined with the water-in-oil emulsion (CA) in said composition (F) as defined previously include copolymers of AMPS and alkyl acrylates in which the carbon chain contains between four and 30 carbon atoms and more particularly between ten and 30 carbon atoms, and linear, branched or crosslinked terpolymers of at least one monomer bearing a free, partially salified or totally salified acid function with at least one neutral monomer and at least one monomer of the formula (XXX):

CH2═C(R′3)—C(═O)—[CH2—CH2—O]n′-R′4 (XXX)

in which R′3 represents a hydrogen atom or a methyl radical and R′4 represents a linear or branched alkyl radical containing from eight to 30 carbon atoms, and n′ represents a number greater than or equal to one and less than or equal to 50.

Examples of thickeners and/or gelling agents that may be combined with the water-in-oil emulsion (CA) in said composition (F) as defined previously include polysaccharides consisting solely of monosaccharides, such as glucans or glucose homopolymers, glucomannoglucans, xyloglycans, galactomannans in which the degree of substitution (DS) of the D-galactose units in the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

Examples of thickeners and/or gelling agents that may be combined with the water-in-oil emulsion (CA) in said composition (F) as defined previously include polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates, karaya gum exudates, and glucosaminoglycans.

Examples of thickeners and/or gelling agents that may be combined with the water-in-oil emulsion (CA) in said composition (F) as defined previously include cellulose, cellulose derivatives such as methylcellulose, ethylcellulose, hydroxypropylcellulose, silicates, starch, hydrophilic starch derivatives, and polyurethanes.

Examples of stabilizers that may be combined with the water-in-oil emulsion (CA) in said composition (F) as defined previously include monocrystalline waxes and more particularly ozokerite, mineral salts such as sodium chloride or magnesium chloride, and silicone polymers such as polysiloxane polyalkyl polyether copolymers.

Examples of solvents that may be combined with the water-in-oil emulsion (CA) in said composition (F) as defined previously include water, organic solvents, for example glycerol, diglycerol, glycerol oligomers, ethylene glycol, propylene glycol, butylene glycol, propane-1,3-diol, propane-1,2-diol, hexylene glycol, diethylene glycol, xylitol, erythritol, sorbitol, water-soluble alcohols such as ethanol, isopropanol or butanol, and mixtures of water and said organic solvents.

Examples of spring or mineral waters that may be combined with the water-in-oil emulsion (CA) in said composition (F) as defined previously include spring or mineral waters having a mineral content of at least 300 mg/I, in particular Avene water, Vittel water, Vichy basin water, Uriage water, La Roche Posay water, La Bourboule water, Enghien-les-bains water, Saint-Gervais-les-bains water, Néris-les-bains water, Allevard-les-bains water, Digne water, Maizieres water, Neyrac-les-bains water, Lons le Saunier water, Rochefort water, Saint Christau water, Fumades water, and Tercis-les-bains water.

Examples of hydrotropic agents that may be combined with the water-in-oil emulsion (CA) in said composition (F) as defined previously include xylene sulfonates, cumene sulfonates, hexyl polyglucoside, 2-ethylhexyl polyglucoside, and n-heptyl polyglucoside.

Examples of emulsifying surfactants that may be combined with the water-in-oil emulsion (CA) in said composition (F) as defined previously include nonionic surfactants, anionic surfactants, and cationic surfactants.

Examples of nonionic emulsifying surfactants that may be combined with the water-in-oil emulsion (CA) in said composition (F) as defined previously include fatty acid esters of sorbitol, for example the products sold under the names Montane™ 40, Montane™ 60, Montane™ 70, Montane™ 80, and Montane™ 85; compositions comprising glyceryl stearate and stearic acid ethoxylated with between 5 mol and 150 mol of ethylene oxide, for example the composition comprising stearic acid ethoxylated with 135 mol of ethylene oxide and the glyceryl stearate sold under the name Simulsol™ 165; mannitan esters, ethoxylated mannitan esters; sucrose esters; methyl glucoside esters; alkyl polyglycosides containing a linear or branched, saturated or unsaturated aliphatic radical containing from 14 to 36 carbon atoms, such as tetradecyl polyglucoside, hexyldecyl polyglucoside, octadecyl polyglucoside, hexyldecyl polyxyloside, octadecyl polyxyloside, eicosyl polyglucoside, dodecosyl polyglucoside, 2-octyldodecyl polyxyloside, 12-hydroxystearyl polyglucoside, compositions of linear or branched, saturated or unsaturated fatty alcohols containing from 14 to 36 carbon atoms and of alkyl polyglycosides as described previously, for example the compositions sold under the names Montanov™ 68, Montanov™ 14, Montanov™ 82, Montanov™ 202, Montanov™ S, Montanov™ WO18, Montanov™ L, Fluidanov™ 20X, and Easynov™.

Examples of anionic surfactants that may be combined with the water-in-oil emulsion (CA) in said composition (F) as defined previously include glyceryl stearate citrate, cetearyl sulfate, soaps such as sodium stearate or triethanolammonium stearate, and N-acyl derivatives of salified amino acids, for example stearoyl glutamate.

Examples of cationic emulsifying surfactants that may be combined with the water-in-oil emulsion (CA) in said composition (F) as defined previously include amine oxides, quaternium-82, and the surfactants described in patent application WO 96/00719 and mainly those in which the fatty chain contains at least 16 carbon atoms.

Examples of opacifiers and/or pearlizing agents that may be combined with the water-in-oil emulsion (CA) in said composition (F) as defined previously include sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols containing from 12 to 22 carbon atoms.

Examples of texturing agents that may be combined with the water-in-oil emulsion (CA) in said composition (F) as defined previously include N-acylamino acid derivatives, for example the lauroyl lysine sold under the name Aminohope™ LL, the octenyl starch succinate sold under the name Dryflo™, the myristyl polyglucoside sold under the name Montanov™ 14, cellulose fibers, cotton fibers, chitosan fibers, talc, sericite, and mica.

Examples of deodorants that may be combined with the water-in-oil emulsion (CA) in said composition (F) as defined previously include alkali metal silicates, zinc salts, such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; decane-1,2-diol, propane-1,3-diol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and glycol, such as the complex of aluminum chlorohydrate and propylene glycol, the complex of aluminum dichlorohydrate and propylene glycol, the complex of aluminum sesquichlorohydrate and propylene glycol, the complex of aluminum chlorohydrate and polyethylene glycol, the complex of aluminum dichlorohydrate and polyethylene glycol, or the complex of aluminum sesquichlorohydrate and polyethylene glycol.

Examples of oils that may be combined with the water-in-oil emulsion (CA) in said composition (F) as defined previously include mineral oils, such as liquid paraffin, liquid petroleum jelly, isoparaffins or white mineral oils; oils of animal origin, such as squalene or squalane; vegetable oils, such as phytosqualane, sweet almond oil, coconut kernel oil, castor oil, jojoba oil, olive oil, rapeseed oil, peanut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils derived from flowers or vegetables or ethoxylated vegetable oils; synthetic oils, such as fatty acid esters, for example butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate or isocetyl lanolate, fatty acid monoglycerides, diglycerides and triglycerides, such as glycerol triheptanoate, alkyl benzoates, hydrogenated oils, poly(α-olefins), polyolefins, such as poly(isobutane), synthetic isoalkanes, such as isohexadecane or isododecane, or perfluorinated oils; silicone oils, such as dimethylpolysiloxanes, methylphenylpolysiloxanes, silicones modified by amines, silicones modified by fatty acids, silicones modified by alcohols, silicones modified by alcohols and fatty acids, silicones modified by polyether groups, epoxy-modified silicones, silicones modified by fluorinated groups, cyclic silicones, and silicones modified by alkyl groups. In the present patent application, “oils” is understood as meaning compounds and/or mixtures of compounds that are insoluble in water and liquid in appearance at a temperature of 25° C.

Examples of waxes that may be combined with the water-in-oil emulsion (CA) in said composition (F) as defined previously include beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes, plant waxes, fatty alcohols and fatty acids that are solid at room temperature, and glycerides that are solid at room temperature. In the present patent application, “waxes” is understood as meaning compounds and/or mixtures of compounds that are insoluble in water and liquid in appearance at a temperature of greater than or equal to 45° C.

Examples of active ingredients that may be combined with the water-in-oil emulsion (CA) in said composition (F) as defined previously include vitamins and derivatives thereof, in particular esters thereof, for example retinol (vitamin A) and esters thereof (for example retinyl palmitate), ascorbic acid (vitamin C) and esters thereof, sugar derivatives of ascorbic acid (such as ascorbyl glucoside), tocopherol (vitamin E) and esters thereof (such as tocopheryl acetate), vitamin B3 or B10 (niacinamide and derivatives thereof); compounds having a lightening or depigmenting action on the skin, such as the ω-undecylenoyl phenylalanine sold under the name Sepiwhite™ MSH, Sepicalm™ VG, the glycerol monoester and/or glycerol diester of ω-undecylenoyl phenylalanine, ω-undecylenoyl dipeptides, arbutin, kojic acid, hydroquinone; compounds having a soothing action, especially Sepicalm™ S, allantoin and bisabolol; anti-inflammatory agents; compounds having a moisturizing action, such as urea, hydroxyureas, glycerol, polyglycerols, glycerol glucoside, diglycerol glucoside, polyglyceryl glucosides, xylityl glucoside; polyphenol-rich plant extracts, such as grape extracts, pine extracts, wine extracts or olive extracts; compounds having a slimming or lipolytic action, such as caffeine or its derivatives, Adiposlim™, Adipoless™, fucoxanthin; N-acylated proteins; N-acylated peptides, such as Matrixyl™; N-acylated amino acids; partial hydrolyzates of N-acylated proteins; amino acids; peptides; total hydrolyzates of proteins; soybean extracts, for example Raffermine™; wheat extracts, for example Tensine™ or Gliadine™; plant extracts, such as tannin-rich plant extracts, isoflavone-rich plant extracts or terpene-rich plant extracts; extracts of freshwater or marine algae; marine plant extracts; marine extracts in general, such as corals; essential waxes; bacterial extracts; ceramides; phospholipids; compounds having an antimicrobial action or a purifying action, such as Lipacide™ CBG, Lipacide™ UG, Sepicontrol™ A5, Octopirox™ or Sensiva™ SC50; compounds having an energizing or stimulating property, such as Physiogenyl™, panthenol and derivatives thereof, such as Sepicap™ MP; antiaging active ingredients, such as Sepilift™ DPHP, Lipacide™ PVB, Sepivinol™, Sepivital™, Manoliva™, Phyto-Age™, Timecode™; Survicode™; antiphotoaging active ingredients; active ingredients that protect the integrity of the dermoepidermal junction; active ingredients that increase the synthesis of components of the extracellular matrix, such as collagen, elastins or glycosaminoglycans; active ingredients having a favorable action on chemical cell communication, such as cytokines, or physical cell communication, such as integrins; active ingredients that create a sensation of “warmth” on the skin, such as activators of cutaneous microcirculation (such as nicotinic acid derivatives) or products that create a sensation of “coolness” on the skin (such as menthol and derivatives); active ingredients that improve cutaneous microcirculation, for example venotonics; draining active ingredients; active ingredients having a decongestant action, such as extracts of Ginkgo biloba, ivy, horse chestnut, bamboo, Ruscus, butcher's broom, Centella asiatica, fucus, rosemary or willow; agents for tanning or browning the skin, for example dihydroxyacetone (DHA), erythrulose, mesotartaric aldehyde, glutaraldehyde, glyceraldehyde, alloxan or ninhydrin, plant extracts, for example extracts of red woods of the genus Pterocarpus and of the genus Baphia, such as Pteropcarpus santalinus, Pterocarpus osun, Pterocarpus soyauxii, Pterocarpus erinaceus, Pterocarpus indicus or Baphia nitida, such as those described in European patent application EP 0 971 683; agents known for their action in facilitating and/or accelerating tanning and/or browning of human skin, and/or for their action in coloring human skin, for example carotenoids (and more particularly β-carotene and γ-carotene), the product sold under the brand name Carrot Oil (INCI name: Daucus carrota, Helianthus annuus sunflower oil) by Provital, which contain carotenoids, vitamin E, and vitamin K; tyrosine and/or derivatives thereof, known for their effect on the acceleration of the tanning of human skin in combination with exposure to ultraviolet radiation, for example the product sold under the brand name SunTan Accelerator™ by Provital, which contains tyrosine and riboflavins (vitamin B), the complex of tyrosine and of tyrosinase sold under the brand name Zymo Tan Complex by Zymo Line, the product sold under the brand name MelanoBronze™ (INCI name: Acetyl Tyrosine, monk's pepper extract (Vitex agnus-castus)) by Mibelle, which contains acetyltyrosine, the product sold under the brand name Unipertan VEG-24/242/2002 (INCI names: Butylene Glycol, Acetyl Tyrosine, Hydrolyzed Vegetable Protein, and Adenosine Triphosphate) by Unipex, the product sold under the brand name Try-Excell™ (INCI names: Oleoyl Tyrosine, Luffa Cylindrica Seed Oil, and Oleic Acid) by Sederma, which contains extracts of marrow seed (or loofah oil), the product sold under the brand name Actibronze™ (INCI names: Hydrolyzed Wheat Protein, Acetyl Tyrosine, and Copper Gluconate) by Alban Muller, the product sold under the brand name Tyrostan™ (INCI name: Potassium Caproyl Tyrosine) by Synerga, the product sold under the brand name Tyrosinol (INCI names: Sorbitan Isostearate, Glyceryl Oleate, Caproyl Tyrosine) by Synerga, the product sold under the brand name InstaBronze™ (INCI names: Dihydroxyacetone, Acetyl Tyrosine, and Copper Gluconate) by Alban Muller, the product sold under the brand name Tyrosilane (INCI names: Methylsilanol and Acetyl Tyrosine) by Exymol; peptides known for their effect in activating melanogenesis, for example the product sold under the brand name Bronzing SF Peptide powder (INCI names: Dextran and Octapeptide-5) by Infinitec Activos, the product sold under the brand name Melitane (INCI names: Glycerin, Aqua, Dextran, and Acetyl hexapeptide-1) comprising the acetyl hexapeptide-1 known for its alpha-MSH agonist action, the product sold under the trade name Melatimes Solutions™ (INCI names: Butylene Glycol, Palmitoyl Tripeptide-40) by Lipotec, sugars and sugar derivatives, for example the product sold under the brand name Tanositor (INCI name: Inositol) by Provital, the product sold under the brand name Thalitan™ (or Phycosaccharide™ AG) by CODIF International (INCI names: Aqua, Hydrolyzed Algin (Laminaria digitata), Magnesium Sulfate, and Manganese Sulfate) containing an oligosaccharide of marine origin (guluronic acid and mannuronic acid chelated with magnesium and manganese ions), the product sold under the brand name Melactiva™ (INCI names: Maltodextrin, Mucuna Pruriens Seed Extract) by Alban Muller, compounds rich in flavonoids, for example the product sold under the brand name Biotanning (INCI name: Hydrolyzed Citrus Aurantium Dulcis Fruit Extract) by Silab and known to be rich in lemon flavonoids (of hesperidin type); agents intended for the treatment of head and/or body hair, for example agents for protecting the melanocytes of the hair follicle, which are intended to protect said melanocytes against cytotoxic agents responsible for the senescence and/or apoptosis of said melanocytes, such as mimetics of the activity of DOPAchrome tautomerase selected from those described in the European patent application published under the number EP1515688 A2, synthetic substances that mimic SOD, for example manganese complexes, antioxidant compounds, for example cyclodextrin derivatives, silicon compounds derived from ascorbic acid, lysine pyrrolidonecarboxylate or arginine pyrrolidonecarboxylate, combinations of mono- and diesters of cinnamic acid and of vitamin C, and more generally those mentioned in the European patent application published under the number EP 1 515 688 A2.

Examples of antioxidant agents that may be combined with the water-in-oil emulsion (CA) in said composition (F) as defined previously include EDTA and salts thereof, citric acid, tartaric acid, oxalic acid, BHA (butylated hydroxyanisole), BHT (butylated hydroxytoluene), tocopherol derivatives such as tocopherol acetate, and mixtures of antioxidant compounds such as Dissolvine™ GL 47S sold by Akzo Nobel under the INCI name Tetrasodium Glutamate Diacetate.

Examples of sunscreens that may be combined with the water-in-oil emulsion (CA) in said composition (F) as defined previously include those listed in the amended Cosmetics Directive 76/768/EEC Annex VII.

Organic sunscreens that may be combined with the water-in-oil emulsion (CA) in said composition (F) as defined previously include the family of benzoic acid derivatives, such as para-aminobenzoic acids (PABA), especially monoglycerol esters of PABA, ethyl esters of N,N25-propoxy PABA, ethyl esters of N,N-diethoxy PABA, ethyl esters of N,N-dimethyl PABA, methyl esters of N,N-dimethyl PABA or butyl esters of N,N-dimethyl PABA; the family of anthranilic acid derivatives, such as homomenthyl N-acetylanthranilate; the family of salicylic acid derivatives, such as amyl salicylate, homomenthyl salicylate, ethylhexyl salicylate, phenyl salicylate, benzyl salicylate or p-isopropylphenyl salicylate; the family of cinnamic acid derivatives, such as ethylhexyl cinnamate, ethyl 4-isopropylcinnamate, methyl 2,5-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate or mono(2-ethylhexanoyl)glyceryl di(para-methoxycinnamate); the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2,5-carboxylate, 2-hydroxy-4-(n-octyloxy)benzophenone, 4-hydroxy-3-carboxybenzophenone; 3-(4′-methylbenzylidene)-d,l-camphor, 3-benzylidene-d,l-camphor, camphor benzalkonium methosulfate; urocanic acid, ethyl urocanate; the family of sulfonic acid derivatives, such as 2-phenylbenzimidazole-5-sulfonic acid and salts thereof; the family of triazine derivatives, such as hydroxyphenyl triazine, ethylhexyloxyhydroxyphenyl-4-methoxyphenyltriazine, 2,4,6-trianilino(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis(2-ethylhexyl) ester of benzoic acid, 2-phenyl-5-methylbenzoxazole, 2,2′-hydroxy-5-methylphenylbenzotriazole, 2-(2′-hydroxy-5′-(t-octyl)phenyl)benzotriazole, 2-(2′-hydroxy-5′-methylphenyl)benzotriazole; dibenzalazine; dianisoylmethane, 4-methoxy-4″-t-butylbenzoylmethane; 5-(3,3-dimethyl-2-norbornylidene)-3-pentan-2-one; the family of diphenylacrylate derivatives, such as 2-ethylhexyl 2-cyano-3,3-diphenyl-2-propenoate or ethyl 2-cyano-3,3-diphenyl-2-propenoate; or the family of polysiloxanes, such as benzylidene siloxane malonate.

Mineral sunscreens, also known as “mineral sunblocks”, that may be combined with the water-in-oil emulsion (CA) in said composition (F) as defined previously include titanium oxides, zinc oxides, cerium oxide, zirconium oxide, yellow, red or black iron oxides, and chromium oxides. These mineral sunblocks may or may not be micronized, may or may not have been subjected to surface treatments, and may optionally be in the form of aqueous or oily predispersions.

### EXAMPLES

The examples that follow illustrate the invention without, however, limiting it.

### Example 1: Preparation of a Concentrated Water-In-Oil Emulsion of the Invention, Composed of C15-19 Alkanes as the Fatty Phase and Sodium PGGA Crosslinked With Butane-1,4-diol Diglycidyl Ether in the Aqueous Phase (pH=5.5 to 6.0)

The synthesis process comprises the following steps:
Step a): Production of a sodium PGGA gel:
   110 grams of demineralized water is placed under mechanical stirring provided by a Rayneri™ stirrer equipped with a deflocculating turbine.
   30 grams of sodium PGGA sold under the brand name “Cosmetic Grade Sodium PolyGammaGlutamate” by Lubon is slowly added to the vortex.
Step b): Adjustment of the pH of the reaction medium to between 5.5 and 6 at a temperature of 20° C. using 5 M HCl solution.
Step c): Addition of 0.45 grams of butane-1,4-diol diglycidyl ether (sold under the name Erisys™ GE 21 by Emerald) to the aqueous phase prepared in step b).
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of C15-19 Alkane (sold under the name Emogreen™ L19 by SEPPIC).
   Weigh out 30 grams of C11-12 Isoparaffin (sold under the name Isopar H by ExxonMobil Chemical).

Homogenize the organic phase by mixing using a magnetic stirrer and a magnetic stirrer bar.
Step e): Pre-emulsification: Addition of the organic phase prepared in step d) to the aqueous phase prepared in step c) with mechanical stirring provided by a Rayneri™ stirrer equipped with a deflocculating turbine.
Step f): Shear emulsification provided by a rotor-stator system by a Silverson™ L4RT mixer for 2 minutes at a speed of 7500 rpm.
Step g): Vacuum distillation, in a reactor under reduced pressure, of the light oil and the water.
Step h): Addition of an oil-in-water surfactant to the concentrated emulsion obtained in step h): weighing out 8 grams of the concentrated emulsion and adding 2 grams of Polyglyceryl-6 Laurate.

Stirring the mixture to obtain a composition (E1).

### Example 2: Preparation of a Concentrated Water-In-Oil Emulsion of the Invention, Composed of Ethylhexyl Palmitate as the Fatty Phase and Sodium PGGA Crosslinked With butane-1,4-diol diglycidyl ether in the Aqueous Phase (pH=5.5 to 6.0)

The synthesis process comprises the following steps:
Step a): Production of a sodium PGGA gel:
   120 grams of demineralized water is placed in a beaker under stirring provided by a Rayneri™ mechanical stirrer equipped with a deflocculating turbine.
   20 grams of sodium PGGA sold under the brand name “Cosmetic Grade Sodium PolyGammaGlutamate” by Lubon is slowly added to the vortex.
Step b): Addition of 0.50 grams of butane-1,4-diol diglycidyl ether (sold under the name Erisys™ GE 21 by Emerald) to the aqueous phase prepared in step a).
Step c): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).

Homogenize the organic phase by mixing using a magnetic stirrer and a magnetic stirrer bar.
Step d): Pre-emulsification: Addition of the organic phase prepared in step c) to the aqueous phase prepared in step b) with mechanical stirring provided by a Rayneri™ stirrer equipped with a deflocculating turbine.
Step e): Shear emulsification with a Silverson™ L4RT rotor-stator device for 2 minutes at a speed of 7500 rpm.
Step f): Vacuum distillation, in a reactor under reduced pressure, of the light oil and the water.
Step g): Addition of an oil-in-water surfactant to the concentrated emulsion obtained in step f): weighing out 8 grams of the concentrated emulsion obtained in step f) and adding 2 grams of polyglyceryl-6 laurate.

The mixture is homogenized at room temperature with mechanical stirring at moderate speed to obtain composition (E2).

### Example 3: Preparation of a Concentrated Water-In-Oil Emulsion of the Invention, Composed of a Mixture of Ethylhexyl Palmitate/C15-19 Alkanes as the Fatty Phase and Sodium PGGA Crosslinked With butane-1,4-diol diglycidyl ether in the Aqueous Phase (pH=5.5 to 6.0)

The synthesis process comprises the following steps:
Step a): Production of a sodium PGGA gel:
   100 grams of demineralized water is placed in a beaker under stirring provided by a Rayneri™ mechanical stirrer equipped with a deflocculating turbine.
   30 grams of PGGA sold under the brand name “Cosmetic Grade Sodium PolyGammaGlutamate” by Lubon is slowly added to the vortex.
Step b): Adjustment of the pH of the reaction medium to between 5.5 and 6 at a temperature of 20° C. using 5 M HCl solution.
Step c): Addition of 0.75 grams of butane-1,4-diol diglycidyl ether (sold under the name Erisys™ GE 21 by Emerald) to the aqueous phase prepared in step b).
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 10 grams of ethylhexyl palmitate (sold under the name DUB PO by Stéarinerie Dubois).
   Weigh out 10 grams of C15-19 Alkane (sold under the name Emogreen™ L19 by SEPPIC).
   Weigh out 30 grams of C11-12 Isoparaffin (sold under the name Isopar™ H by ExxonMobil Chemical).

The mixture obtained is stirred by means of a magnetic stirrer and a magnetic stirrer bar.
Step e): Pre-emulsification: Addition of the organic phase prepared in step d) to the aqueous phase prepared in step c) with mechanical stirring using a Rayneri™ stirrer equipped with a deflocculating turbine.
Step f): Emulsification by shear stirring with a rotor-stator device with a Silverson™ L4RT stirrer for 2 minutes at 7500 rpm.
Step g): Vacuum distillation, in a reactor under reduced pressure, of the light oil and the water.
Step h): Addition of an oil-in-water surfactant to the concentrated emulsion obtained in step g): weighing out 8 grams of the concentrated emulsion obtained in step g) and adding 2 grams of polyglyceryl-6 laurate.

The mixture is homogenized at room temperature with mechanical stirring at moderate speed to obtain composition (E3).

### Example 4: Preparation of a Concentrated Water-In-Oil Emulsion of the Invention, Composed of Ethylhexyl Palmitate as the Fatty Phase and Sodium PGGA Crosslinked With butane-1,4-diol diglycidyl ether in the Organic Phase (pH=5.5 to 6.0) (Crosslinking Agent in Fatty Phase)

The synthesis process comprises the following steps:
Step a): Production of a sodium PGGA gel:
   120 grams of demineralized water is placed in a beaker under mechanical stirring using a Rayneri™ stirrer equipped with a deflocculating turbine.
   20 grams of PGGA sold under the brand name “Cosmetic Grade Sodium PolyGammaGlutamate” by Lubon is slowly added to the vortex.
Step b): Adjustment of the pH of the reaction medium to between 5.5 and 6 at a temperature of 20° C. using 4 M NaOH solution.
Step c): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Weigh out 0.50 grams of butane 1,4-diol diglycidyl ether (sold under the name Erisys GE 21 by Emerald).
   Stir the mixture of ingredients previously weighed out with magnetic stirring using a magnetic stirrer bar.
Step d): Pre-emulsification: Addition of the organic phase prepared in step c) to the aqueous phase prepared in step b) with mechanical stirring using a Rayneri™ mechanical stirrer equipped with a deflocculating turbine.
Step e): Shear emulsification with a stirrer equipped with a Silverson™ L4RT rotor-stator system for 2 min at a speed of 7500 rpm.
Step f): Vacuum distillation, in a reactor under reduced pressure, of the light oil and the water.
Step g): Addition of oil-in-water surfactant to the concentrated emulsion obtained in step f): weighing out 8 grams of the concentrated emulsion and 2 grams of polyglyceryl-6 laurate, which are added to the mixture obtained in step f).

The mixture is homogenized at room temperature with mechanical stirring at moderate speed to obtain composition (E4).

### Example 5: Preparation of a Concentrated Water-In-Oil Emulsion of the Invention, Composed of Ethylhexyl Palmitate as the Fatty Phase and Sodium PGGA Crosslinked With butane-1,4-diol diglycidyl ether in Organic Phase (pH=4)

The synthesis process comprises the following steps:
Step a): Preparation of a sodium PGGA gel:
   120 grams of demineralized water is placed in a beaker under mechanical stirring using a Rayneri™ stirrer equipped with a deflocculating turbine.
   20 grams of PGGA sold under the brand name “Cosmetic Grade Sodium PolyGammaGlutamate” by Lubon is slowly added to the vortex.
Step b): Adjustment of the pH of the reaction medium to 4 at a temperature of 20° C. using 5 M HCl solution.
Step c): Addition of 0.50 grams of butane-1,4-diol diglycidyl ether (sold under the name Erisys™ GE 21 by Emerald) to the aqueous phase prepared in step b).
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Add each of the ingredients to the beaker and stir the mixture with a mechanical stirrer equipped with a magnetic stirrer bar.
Step e): Pre-emulsification: Addition of the organic phase prepared in step d) to the aqueous phase prepared in step c) with mechanical stirring using a Rayneri™ stirrer equipped with a deflocculating turbine.
Step f): Shear emulsification with a Silverson™ L4RT, for 2 minutes at a speed of 7500 rpm.
Step g): Vacuum distillation, in a reactor under reduced pressure, of the light oil and the water.
Step h): Addition of water-in-oil surfactant to the concentrated emulsion obtained in step g): weighing out 8 grams of the concentrated emulsion obtained in step g) and adding 2 grams of polyglyceryl-6 laurate.

The mixture is homogenized at room temperature with mechanical stirring at moderate speed to obtain composition (E5).

### Example 6: Preparation of a Concentrated Water-In-Oil Emulsion of the Invention, Composed of Ethylhexyl Palmitate as the Fatty Phase and Sodium PGGA Crosslinked With butane-1,4-diol diglycidyl ether in the Aqueous Phase (pH=10)

The synthesis process comprises the following steps:
Step a): Preparation of a sodium PGGA gel:
   120 grams of demineralized water is placed under mechanical stirring using a Rayneri™ stirrer equipped with a deflocculating turbine.
   20 grams of PGGA sold under the brand name “Cosmetic Grade Sodium PolyGammaGlutamate” by Lubon is slowly added to the vortex.
Step b): Adjustment of the pH of the reaction medium to 10 at a temperature of 20° C. using 4 M NaOH solution.
Step c): Addition of 0.50 grams of butane-1,4-diol diglycidyl ether (sold under the name Erisys™ GE 21 by Emerald) to the aqueous phase prepared in step b).
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Mix the various ingredients and stir the mixture using a magnetic stirrer equipped with a magnetic stirrer bar.
Step e): Pre-emulsification: Addition of the organic phase prepared in step d) to the aqueous phase prepared in step c) with mechanical stirring using a Rayneri™ stirrer equipped with a deflocculating turbine.
Step f): Shear emulsification with a Silverson™ L4RT stirrer for 2 minutes at a speed of 7500 rpm.
Step g): Distillation under reduced pressure (either with a rotary evaporator+flask or in a vacuum reactor) of the light oil and the water.
Step h): Addition of oil-in-water surfactant to the concentrated emulsion obtained in step g): weighing out 8 grams of the concentrated emulsion obtained in step g) and 2 grams of polyglyceryl-6 laurate.

The mixture is homogenized at room temperature with mechanical stirring at moderate speed to obtain composition (E6).

### Example 7: Preparation of a Concentrated Water-In-Oil Emulsion of the Invention, Composed of Ethylhexyl Palmitate as the Fatty Phase and Sodium PGGA Crosslinked With butane-1,4-diol diglycidyl ether and Lipophilized With C12-C14 Glycidyl Ether in the Aqueous Phase (pH=6.5)

The synthesis process comprises the following steps:
Step a): Preparation of a sodium PGGA gel:
   120 grams of demineralized water is placed in a beaker under mechanical stirring with a Rayneri™ stirrer equipped with a deflocculating turbine.
   20 grams of PGGA sold under the brand name “Cosmetic Grade Sodium PolyGammaGlutamate” by Lubon is slowly added to the vortex.
Step b): Adjustment of the pH of the reaction medium to between 5.5 and 6 at a temperature of 20° C. using 4 M NaOH solution.
Step c): Addition of 0.5 grams of butane-1,4-diol diglycidyl ether (sold under the name Erisys™ GE 21 by Emerald) to the aqueous phase prepared in step b).
Step d): Addition of 2.0 grams of C12-C14 glycidyl ether (sold under the name Erisys™ GE 08 from Emerald) to the aqueous phase prepared in step c).
Step e): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Stir the mixture of ingredients with a magnetic stirrer equipped with a magnetic stirrer bar.
Step f): Pre-emulsification: Addition of the organic phase prepared in step e) to the aqueous phase prepared in step d) with mechanical stirring with a Rayneri™ stirrer equipped with a deflocculating turbine.
Step g): Shear emulsification with a Silverson™ L4RT stirrer for 2 minutes at a speed of 7500 rpm.
Step h): Vacuum distillation, in a reactor under reduced pressure, of the light oil and the water.
Step i): Addition of oil-in-water surfactant to the concentrated emulsion obtained in step g): weighing out 8 grams of the concentrated emulsion obtained in step h) and 2 grams of polyglyceryl-6 laurate.

The mixture is homogenized at room temperature with mechanical stirring at moderate speed to obtain composition (E7).

### Example 8: Preparation of a Concentrated Water-In-Oil Emulsion of the Invention, Composed of Ethylhexyl Palmitate as the Fatty Phase and Sodium PGGA Crosslinked With Trimethylolethane Triglycidyl Ether in the Aqueous Phase (pH=6.0)

The synthesis process comprises the following steps:
Step a): Preparation of a sodium PGGA gel:
   120 grams of demineralized water is placed under mechanical stirring with a Rayneri™ stirrer equipped with a deflocculating turbine.
   20 grams of PGGA sold under the brand name “Cosmetic Grade Sodium PolyGammaGlutamate” by Lubon is slowly added to the vortex.
Step b): Adjustment of the pH of the reaction medium to between 5.5 and 6.0 at a temperature of 20° C. using 4 M NaOH solution.
Step c): Addition of 0.5 grams of trimethylolethane triglycidyl ether (sold under the name Erisys™ GE 31 by Emerald) to the aqueous phase prepared in step b)
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Stir the mixture of all the ingredients with a magnetic stirrer equipped with a magnetic stirrer bar.
Step e): Pre-emulsification: Addition of the organic phase prepared in step d) to the aqueous phase prepared in step c) with mechanical stirring using a Rayneri™ stirrer equipped with a deflocculating turbine.
Step f): Shear emulsification with a Silverson™ L4RT stirrer for 2 minutes at a speed of 7500 rpm.
Step g): Vacuum distillation, in a reactor under reduced pressure, of the light oil and the water.
Step h): Addition of oil-in-water surfactant to the concentrated emulsion obtained in step g): weighing out 8 grams of the concentrated emulsion obtained in step f) and 2 grams of polyglyceryl-6 laurate.

The mixture is homogenized at room temperature with mechanical stirring at moderate speed to obtain composition (E8).

### Example 9: Preparation of a Crosslinked PGA(Na) Concentrated Inverse Latex of the Invention in Octyl Palmitate

The synthesis process comprises the following steps:
Step a): Production of a sodium PGGA gel with a Rayneri™ mechanical stirrer equipped with a deflocculating turbine:
   110 grams of demineralized water is placed under stirring in a beaker and stirred with a Rayneri™ mechanical stirrer equipped with a deflocculating turbine.
   30 grams of PGGA sold under the brand name “Cosmetic Grade Sodium PolyGammaGlutamate” by Lubon is slowly added to the vortex.
Step b): Adjustment of the pH of the reaction medium to 5.5-6 at a temperature of 20° C. using 5 M HCl solution.
Step c): Addition of 0.72 grams of butane-1,4-diol diglycidyl ether (sold under the name Erisys™ GE 21 by Emerald) to the aqueous phase prepared in step b).
Step d): Preparation of the organic phase in a 100 gram beaker:
   Weigh out 5 grams of sorbitan isostearate (sold under the name Montane™ 70 by SEPPIC)
   Weigh out 3 grams of a mixture consisting of tall oil diethanolamide sold under the brand name Simaline™ IE 200 by SEPPIC.
   Weigh out 2 grams of a polymeric surfactant sold under the brand name Hypermer™ 6212 by Croda.
   Weigh out 50 grams of C11-12 Isoparaffin (sold under the name Isopar H by ExxonMobil Chemical).
   Stir the mixture of all the ingredients using a magnetic stirrer equipped with a magnetic stirrer bar.
Step e): Pre-emulsification: Addition of the organic phase prepared in step d) to the aqueous phase prepared in step c) with mechanical stirring using a Rayneri™ stirrer equipped with a deflocculating turbine.
Step f): Shear emulsification with a Silverson™ L4RT mechanical stirrer for 2 minutes at a speed of 7500 rpm.
Step g): Vacuum distillation, in a reactor under reduced pressure, of the light oil and the water.
Step h): Addition of oil-in-water surfactant to the concentrated emulsion obtained in step g): weighing out 8 grams of the concentrated emulsion and 1 gram of polysorbate 80 (sold under the name Montanox™ 80 by SEPPIC).

The mixture is homogenized at room temperature with mechanical stirring at moderate speed to obtain composition (E9).

### Example 10: Preparation of a Concentrated Water-In-Oil Emulsion of the Invention, Composed of Ethylhexyl Palmitate as the Fatty Phase and Sodium PGGA Crosslinked With Ethylene Glycol Diglycidyl Ether (EGDGE) in the Aqueous Phase (pH=6.0)

The synthesis process comprises the following steps:
Step a): Preparation of a sodium PGGA gel:
   130 grams of demineralized water is placed under mechanical stirring with a Rayneri™ stirrer equipped with a deflocculating turbine.
   10 grams of PGGA sold under the brand name “Cosmetic Grade Sodium PolyGammaGlutamate” by Lubon is slowly added to the vortex.
Step b): Adjustment of the pH of the reaction medium to between 5.5 and 6 at a temperature of 20° C. using 4 M NaOH solution.
Step c): Addition of 0.25 grams of ethylene glycol diglycidyl ether (sold under the name Erisys™ EGDGE by Emerald) to the aqueous phase prepared in step b)
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Stir the mixture consisting of all the above ingredients with a magnetic stirrer equipped with a magnetic stirrer bar.
Step e): Pre-emulsification: Addition of the organic phase prepared in step d) to the aqueous phase prepared in step c) with mechanical stirring using a Rayneri™ stirrer equipped with a deflocculating turbine.
Step f): Shear emulsification with a Silverson™ L4RT stirrer for 2 minutes at a speed of 7500 rpm.
Step g): Vacuum distillation, in a reactor under reduced pressure, of the light oil and the water.
Step h): Addition of oil-in-water surfactant to the concentrated emulsion obtained in step f): weighing out 8 grams of the concentrated emulsion obtained in step g) and 2 grams of polyglyceryl-6 laurate.

The mixture is homogenized at room temperature with mechanical stirring at moderate speed to obtain composition (E10).

### Example 11: Preparation of a Concentrated Water-In-Oil Emulsion of the Invention, Composed of Ethylhexyl Palmitate as the Fatty Phase and Sodium PGGA Crosslinked With butane-1,4-diol diglycidyl ether in the Aqueous Phase (pH=6.0)

The synthesis process comprises the following steps:
Step a): Preparation of a sodium PGGA gel:
   100 grams of demineralized water is placed under mechanical stirring with a Rayneri™ stirrer equipped with a deflocculating turbine.
   40 grams of PGGA sold under the brand name “Cosmetic Grade Sodium PolyGammaGlutamate” by Lubon is slowly added to the vortex.
Step b): Adjustment of the pH of the reaction medium to between 5.5 and 6 at a temperature of 20° C. using 4 M NaOH solution.
Step c): Addition of 0.80 grams of butane-1,4-diol diglycidyl ether (sold under the name Erisys™ GE 21 by Emerald) to the aqueous phase prepared in step b).
Step d): Preparation of the organic phase in a 100 gram beaker:
   Weigh out 5 grams of sorbitan oleate (sold under the name Montane™ 80 VG by SEPPIC).
   Weigh out 5 grams of polyglyceryl-2 dipolyhydroxystearate (sold under the name Dehymuls™ PGPH by BASF).
   Weigh out 20 grams of ethylhexyl palmitate (sold under the name DUB PO by Stéarinerie Dubois).
   Weigh out 30 grams of C11-12 Isoparaffin (sold under the name Isopar H by ExxonMobil Chemical).
   Stir the mixture consisting of all the weighed ingredients with a magnetic stirrer equipped with a magnetic stirrer bar.
Step e): Pre-emulsification: Addition of the organic phase prepared in step d) to the aqueous phase prepared in step c) with mechanical stirring using a Rayneri stirrer equipped with a deflocculating turbine.
Step f): Shear emulsification with a Silverson L4RT stirrer for 2 minutes at a speed of 7500 rpm.
Step g): Vacuum distillation, in a reactor under reduced pressure, of the light oil and the water.
Step h): Addition of oil-in-water surfactant to the concentrated emulsion obtained in step g): weighing out 8 grams of the concentrated emulsion obtained in step g) and 2 grams of polyglyceryl-6 laurate. Stirring

The mixture is homogenized at room temperature with mechanical stirring at moderate speed to obtain composition (E11).

Evaluation of compositions (E1) to (E11) of the invention. The compositions (E1) to (E11) of the invention are evaluated as described below:
Weigh out 192 grams of water in a 400 ml high-sided beaker.
Add with mechanical stirring using a Rayneri stirrer equipped with a deflocculating turbine 8 grams of compositions (E1) to (E11).
Allow to stir until a homogeneous gel is obtained.
Measure the dynamic viscosity of the homogeneous gels using a Brookfield RVT viscometer at a speed of 5 rpm, selecting the appropriate spindle.
Add 0.1% by weight of sodium chloride to the gel previously obtained, and stir with a Rayneri mechanical stirrer equipped with a deflocculating turbine.
Then measure the dynamic viscosity of this novel gel using a Brookfield RVT viscometer at a speed of 5 rpm, selecting the appropriate spindle.

The results are collated in Table 1 below.

TABLE 1

| | | | | Gel viscosity |
|---|---|---|---|---|
| | Gel viscosity at 4% by | Gel viscosity at 4% by | | obtained in step a) of |
| | weight of | weight of composition + | Equivalent % | the preparation |
| | composition | 0.1% by weight of NaCl | by weight of | process |
| | (Brookfield ™ RVT, | (Brookfield ™ RVT, | polymeric | (Brookfield ™ RVT, |
| | speed 5) | speed 5) | active | speed 5) |
| Composition | spindle (x) | spindle (x) | substance | spindle (x) |
| Control test | 2% gel = 176 mPa · s | 2% gel + 0.1% | 2% | 2% gel = 128 mPa · s |
| “Cosmetic grade | (spindle 2) | NaCl = 128 mPa · s | | (spindle 2) |
| sodium PGGA” | | (spindle 2) | | |
| sold by Lubon | | | | |
| Composition | 76 200 mPa · s | 73 000 mPa · s | 2% | 18 120 mPa · s |
| (E1) | (spindle 6) | (spindle 6) | | (spindle 3) |
| Composition | 124 200 mPa · s | 89 400 mPa · s | 1.6% | 5040 mPa · s |
| (E2) | (spindle 6) | (spindle 6) | | (spindle 3) |
| Composition | 91 600 mPa · s | 816 mPa · s | 2% | 18 120 mPa · s |
| (E3) | (spindle 6) | (spindle 3) | | (spindle 3) |
| Composition | 117 200 mPa · s | 58 600 mPa · s | 1.6% | 5040 mPa · s |
| (E4) | (spindle 6) | (spindle 6) | | (spindle 3) |
| Composition | 9820 mPa · s | 8860 mPa · s | 1.6% | 5040 mPa · s |
| (E5) | (spindle 3)* | (spindle 6)* | | (spindle 3) |
| Composition | 131 800 mPa · s | 102 800 mPa · s | 1.6% | 5040 mPa · s |
| (E6) | (spindle 6)* | (spindle 6)* | | (spindle 3) |
| Composition | 167600 mPa · s | 118 400 mPa · s | 1.6% | 5040 mPa · s |
| (E7) | (spindle 6) | (spindle 6) | | (spindle 3) |
| Composition | 9540 mPa · s | 8860 mPa · s | 1.6% | 5040 mPa · s |
| (E8) | (spindle 3) | (spindle 3) | | (spindle 3) |
| Composition | 91 800 mPa · s | 34 600 mPa · s | 1.33% | 18 120 mPa · s |
| (E9) | (spindle 6) | (spindle 6) | | (spindle 3) |
| Composition | 78 000 mPa · s | 600 mPa · s | 1% | 900 mPa · s |
| (E10) | (spindle 6) | (spindle 2) | | (spindle 3) |
| Composition | 74 800 mPa · s | 48 400 mPa · s | 2.28% | 50 000 mPa · s |
| (E11) | (spindle 6) | (spindle 6) | | (spindle 6) |

Dynamic viscosities of aqueous gels obtained with compositions (E1) to (E11)
*Viscosity values of gels at pH 6

Compositions (E1) to (E11) of the invention make it possible to obtain aqueous gels that are thickened by comparison with the aqueous gel obtained from a non-crosslinked sodium gamma-polyglutamate (“control test”).

Thus, at a polymer percentage content of 2% by weight, the aqueous gels obtained with compositions (E1) and (E3) have a viscosity of respectively 76 000 mPa·s and 91 600 mPa·s, whereas the aqueous gel obtained with the non-crosslinked sodium gamma-polyglutamate (“control test”) is characterized by a viscosity of 176 mPa·s.

Similarly, at a polymer percentage content of less than 2% by weight, the aqueous gels obtained with compositions (E2), (E4), (E8), (E9), and (E10) have a viscosity of respectively 124 000 mPa·s, 117 200 mPa·s, 9540 mPa·s, 91 800 mPa·s and 78 000 mPa·s, whereas the aqueous gel obtained with 2% by weight of non-crosslinked sodium gamma-polyglutamate (“control test”) is characterized by a viscosity of 176 mPa·s.

1. A composition in the form of a self-inverting water-in-oil emulsion containing, per 100% of its weight, a content of greater than or equal to 20% by weight of a polymer consisting of monomer units derived from partially or totally salified glutamic acid and of units derived from at least one crosslinking agent bearing at least two glycidyl functions.

2. The composition as claimed in claim 1, wherein the content by weight of the polymer is greater than or equal to 20% and less than or equal to 60%.

3. The composition as claimed in claim 1, wherein the crosslinking agent is selected from the group consisting of:
ethylene glycol diglycidyl ether of the formula 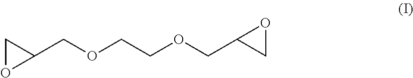 [SMILES: C(COCC1CO1)OCC1CO1]
the compound of the formula 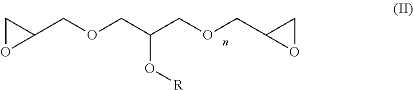
where R represents a hydrogen atom or the glycidyl radical 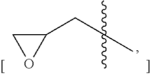 [SMILES: *CC1CO1]
and n represents an integer greater than or equal to one and less than or equal to 10.
propane-1,3-diol diglycidyl ether of the formula 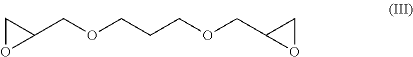 [SMILES: C(COCC1CO1)COCC1CO1]
propane-1,2-diol diglycidyl ether of the formula 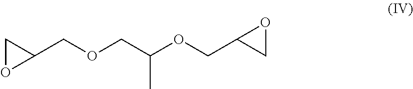
butane-1,4-diol diglycidyl ether of the formula 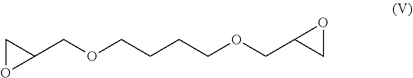 [SMILES: C(CCOCC1CO1)COCC1CO1]
butane-1,2-diol diglycidyl ether of the formula 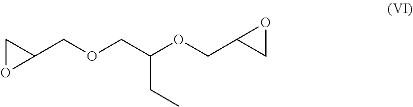
butane-1,3-diol diglycidyl ether of the formula 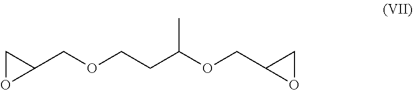
hexane-1,6-diol diglycidyl ether of the formula 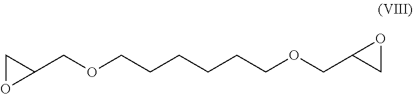 [SMILES: C(CCCOCC1CO1)CCOCC1CO1]
the compound of the formula 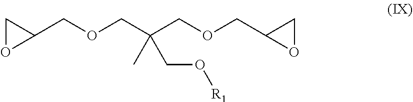
where R1 represents a hydrogen atom or the glycidyl radical 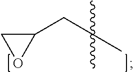
the compound of the formula 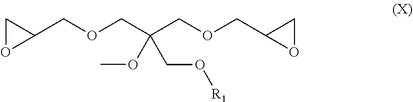
where R1 represents a hydrogen atom or the glycidyl radical 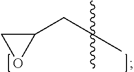
the compound of the formula 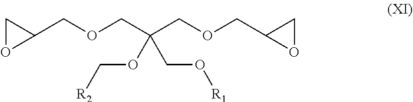
where R1 and R2 each independently represent a hydrogen atom or the glycidyl radical 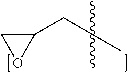
the compound of the formula 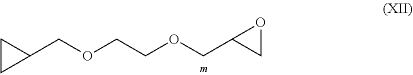
where m represents an integer greater than or equal to 2
the compound of the formula 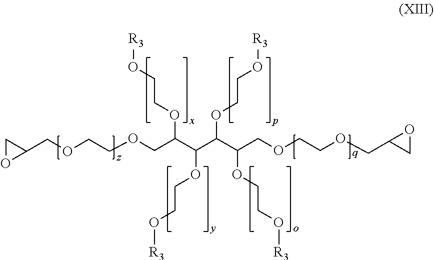
where R3 represents a hydrogen atom or the glycidyl radical 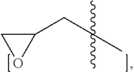
and x, y, z, o, p, and q each independently represent an integer greater than or equal to 2 and less than or equal to 10.

4. The composition as claimed in claim 1, wherein the polymer is gamma-polyglutamic acid in acid form or in partially or totally salified form.

5. The composition as claimed in claim 1, wherein, in the polymer, per 100 mol % of monomer units derived from partially or totally salified glutamic acid, the crosslinking agent represents from 0.5 mol % to 20 mol %.

6. The composition as claimed in claim 1, wherein it has a viscosity of between 100 mPa·s and 10 000 mPa·s.

7. The composition as claimed claim 1, wherein it also comprises a monomer unit derived from the compound of the formula: 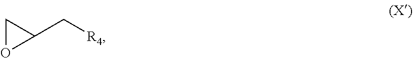
where R4 represents a linear or branched, saturated or unsaturated, functionalized or non-functionalized hydrocarbon radical containing from 6 to 22 carbon atoms.

8. A process for preparing a composition as defined in claim 1, comprising:
a) a step of preparing an aqueous solution comprising partially or totally salified polyglutamic acid, wherein said aqueous solution comprises, per 100% of its weight, between 5% and 70% by weight of partially or totally salified PGA and a crosslinking agent containing at least two glycidyl functions;
b) a step of adjusting the pH of the aqueous solution obtained in step a) to a pH of between 3 and 11;
c) a step of preparing an organic phase comprising at least one volatile oil, at least one other oil that is nonvolatile, and at least one water-in-oil emulsifying surfactant;
d) a step of pre-emulsifying by adding the organic phase obtained in step c) to the aqueous solution obtained in step b) with stirring;
e) a step of emulsifying the pre-emulsion obtained in step d) by homogenizing with stirring;
f) a step of distilling the water and volatile oil contained in the emulsion obtained in step e);
g) a step of adding at least one oil-in-water emulsifying surfactant (S2) so as to obtain the composition.

9. The process as claimed in claim 8, wherein in step a) all of the monomer units making up the gamma-polyglutamic acid are derived from sodium glutamate, potassium glutamate, ammonium glutamate, calcium glutamate, magnesium glutamate or a mixture of these forms.

10. The process as claimed in claim 8, wherein in step a) the crosslinking agent is present in weight proportions of between 0.5% and 10% by weight relative to the weight of polyglutamic acid.

11. The process as claimed in claim 8, wherein in step c) the at least one water-in-oil emulsifying agent is selected from the group consisting of sorbitan esters, polyglycerol esters, alkoxylated polyglycerol esters, polyglycol polyhydroxystearates, polyglyceryl polyhydroxystearates, and alkoxylated polyglyceryl polyhydroxystearates.

12. The process as claimed in claim 8, wherein in step c) the organic solution contains, per 100% of its own weight, between 10% and 30% by weight of at least one water-in-oil emulsifying agent, preferably between 15% and 20% by weight.

13. The process as claimed in claim 8, wherein in step c) the water-in-oil emulsifying agent is a polyglyceryl polyhydroxystearate.

14. The process as claimed in claim 8, wherein in step g) the at least one oil-in-water emulsifying surfactant is selected from the group consisting of a polyethoxylated fatty alcohol, a polyethoxylated hexitan ester, an alkyl polyglycoside, a composition of alkyl polyglycoside and fatty alcohols, a polyglycerol ester, and a composition of polyglycerol ester and polyglycerol.

15. The process as claimed in claim 8, wherein step d) is carried out such that the weight ratio between the aqueous solution and the organic phase is between 90/10 and 10/90, preferably between 20/80 and 40/60.

16. The process as claimed in claim 8, wherein in step a) the aqueous phase also comprises at least one compound of the formula: 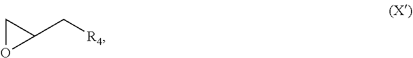
where R4 represents a linear or branched, saturated or unsaturated, functionalized or non-functionalized hydrocarbon radical containing from 6 to 22 carbon atoms.

17. The process as claimed in claim 8, wherein in step e) the homogenization is carried out with mechanical shear stirring.

18. The use of said composition as defined in claim 1 as a thickening and/or emulsifying and/or stabilizing agent for a composition for topical cosmetic use.

19. A cosmetic composition for topical use wherein it contains as thickening agent, per 100% of its total weight, between 0.1% by weight and 10% by weight of said composition as defined in claim 1.
